(19) United States
(12) Patent Application Publication
(10) Pub. No.: US 2015/0362482 A1
(43) Pub. Date: Dec. 17, 2015

(54) METHODS OF DETECTING NEUROTOXIN USING SYNAPTIC ACTIVITY

(71) Applicants: Patrick MCNUTT, (US); Phillip BESKE, Fort Detrick, MD (US); THE UNITED STATES OF AMERICA, as represented by THE SECRETARY OF THE ARMY, on behalf of, Frot Detrick, MD (US)

(72) Inventors: Patrick McNutt, Fort Detrick, MD (US); Phillip Beske, Fort Detrick, MD (US)

(21) Appl. No.: 14/759,083

(22) PCT Filed: Mar. 7, 2014
(86) PCT No.: PCT/US14/22042

§ 371 Date: Jul. 2, 2015

Related U.S. Application Data
(60) Provisional application No. 61/851,599, filed on Mar. 8, 2013.

(51) Int. Cl. G01N 33/50

(52) U.S. Cl. CPC G01N 33/5058; G01N 33/5014; G01N 2333/33

(57) ABSTRACT

The present invention relates to methods of detecting neurotoxin in a sample. The methods of the present invention are highly sensitive and specific, rapid, and clinically relevant with minimal cost. The methods of the present invention use isolated neurons capable of networked synaptic activity, which allows each step of the natural toxin intoxication process to occur, mimicking the clinical manifestation of toxin intoxication. This networked synaptic activity provides rapid and highly sensitive neurotoxin detection. The methods are also directed to detecting neurotoxin neutralizing agents and neurotoxin identity.

### CROSS-REFERENCE TO RELATED APPLICATION

This application claims priority to and the benefit of U.S. Patent Application No. 61/851,599, filed Mar. 8, 2013, the disclosure of which is entirely hereby incorporated herein by reference.

### FIELD OF THE INVENTION

The present invention relates to methods of using synaptic forming neurons, derived from stem cells ex vivo, to detect toxins. The methods of toxin detection include measuring a loss of synaptic activity in these neurons and correlating the loss of synaptic activity with the presence of toxin in the sample.

### BACKGROUND OF THE INVENTION

Neurotoxins are compounds that adversely affect the nervous system. Typically, neurotoxins act by mechanisms that inhibit neuron processes ranging from membrane depolarization to inter-neuron communication. Neurotoxin exposure can result in nervous system arrest or even nervous tissue death. The onset of symptoms upon neurotoxin intoxication can vary between different toxins, being on the order of hours to years. The speed of recover from neurotoxin intoxication increases with timely medical intervention. Thus, sensitive and rapid toxin detection and diagnostic methods are critical.

Due to the nature of neurotoxins, detection methods need to be sensitive, specific, capable of use with complex samples, detect toxin activity, easy to use, and cost effective. For some neurotoxins, suitable detection methods rely on the use of animals and take weeks to provide results. A commonly used animal based detection method is the mouse bioassay. Despite many attempts to replace the use of animals, the mouse bioassay continues to be relied upon because it is capable of modeling all aspects of neurotoxin intoxication including binding, translocation, and activity. Such animal based detection methods have many drawbacks, including, long assay times, the requirement of special animal facilities, the requirement of specially trained staff, substantial variation in results, and high cost.

In the instance of Clostridium botulinum neurotoxins (BoNTs), clinical presentation typically involves a descending flaccid paralysis with consequent respiratory failure 12-96 hours after exposure. After exposure, victims remain clinically asymptomatic until paralysis develops. However, once the toxin internalizes into the pre-synaptic compartment of the neuron, clinically effective therapies to reverse paralysis of intoxicated neuromuscular junctions are not available. Consequently, while post-exposure administration of antitoxin can accelerate clearance of BoNT from the plasma, it is ineffectual once the toxin has entered neurons. Therefore, there is a critical need for a rapid and sensitive method to identify functional toxin following a potential BoNT exposure, before the toxin internalizes into the neurons.

Accordingly, a need exists for toxin detection compositions and methods that are fast, sensitive, specific, easy to use, and cost effective. Further, there is a need for detection methods that do not rely on the death of animals. The present invention provides compositions and methods for toxin, including neurotoxin, detection that are fast, sensitive, specific, easy to use, cost effective, and do not rely on the death of animals.

### SUMMARY OF THE INVENTION

The present invention is related to compositions and methods of detecting toxins using neurons capable of networked synaptic activity. In many aspects, the compositions and methods of the invention use ex vivo networked neuron populations to detect the presence of neurotoxin, providing rapid, sensitive, and clinically relevant assays.

The methods of the present invention include detecting botulinum neurotoxin in a sample. Such methods include identifying a sample potentially exposed to botulinum neurotoxin and contacting it with a networked neuron population ex vivo. The networked synaptic activity of the neuron population is measured at various time points. The measurements are then compared and correlated with neurotoxin detection. The presence of neurotoxin may be detected at femtomolar levels. Also, the presence of neurotoxin may be detected within minutes to hours of contacting the sample with the networked neuron population.

The methods also include detecting synaptic preservation. Such method is useful for identifying neurotoxin intoxication therapies or treatments. The methods include identifying a sample exposed to neurotoxin and contacting it with a networked neuron population ex vivo. The networked synaptic activity of the neuron population is measured at various time points. The measurements are then compared and correlated with synaptic preservation.

In some embodiments, the methods include evaluating the efficacy of neurotoxin neutralizing agents. Such methods include providing a networked neuron population and contacting it with a neurotoxin to produce an intoxicated composition. The intoxicated composition is then contacted with a neutralizing agent. The networked synaptic activity of the neuron population is measured at various time points. The measurements are then compared and correlated with neurotoxin neutralization efficacy.

In some embodiments, the methods include identifying neurotoxins in a sample. Such methods include providing a sample exposed to neurotoxin and contacting it with a networked neuron population to produce an intoxicated composition. The intoxicated composition is then contacted with a neutralizing agent. The networked synaptic activity of the neuron population is measured at various time points. The measurements are then compared and correlated with neurotoxin identity.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the functional characterization of the maturation-dependent development of intrinsic neuronal properties in ESNs. FIG. 1A shows stimulation protocol of voltage-clamp recordings. FIG. 1B shows peak current-voltage plot and representative traces. FIG. 1C shows representative traces of fast-acting and fast inactivating sodium channels. FIG. 1D depicts a schematic stimulation protocol of voltage-clamp. FIG. 1E shows a peak current-voltage plot and representative traces of a delayed rectifier potassium current in DIV2-DIV35 ESNs. FIG. 1F shows representative traces of DIV2-DIV35 ESNs. FIG. 1G shows the maturation-dependent development of a negative resting membrane potential. FIG. 1H shows a schematic stimulation protocol of current-clamp recordings. FIG. 1I shows a representative trace of current-clamp recordings illustrating evoked action potentials in DIV2-DIV35 ESNs.

FIG. 2 shows synaptic development within ESNs. FIG. 2A shows immunocytochemistry performed in ESN cultures from DIV7-DIV35. Axons were labeled with Tau (green), dendrites are labeled with MAP2 (red), pre-synaptic compartments were labeled with synapsin (white), and nuclei were stained with DAPI (blue). FIG. 2B shows a representative trace of spontaneous activity and quantification of events per second from continuous current-clamp. FIG. 2C shows the excitatory post-synaptic currents.

FIG. 3 shows ESN sensitivity to classical BoNT serotypes. FIG. 3A shows the quantification of target SNARE cleavage for BoNT serotypes /A-/G. FIG. 3B shows representative Western blots of target SNARE cleavage at 24 hours following the addition of varying concentrations of BoNT/A-/G. FIG. 3C shows a time- and dose-dependent response of SNAP-25 cleavage following BoNT/A intoxication. FIG. 3D shows a schematic of the experimental design.

FIG. 4 shows the effects of BoNT/A intoxication of ESN synapses in DIV28+ cultures.

FIG. 5 shows the spontaneous activity of DIV28+ESNs post-BoNT/A addition. FIG. 5A shows representative traces of spontaneous activity from current-clamp recordings of DIV28+ following BoNT/A intoxication. FIG. 5B shows quantification of spontaneous activity at indicated duration of BoNT/A exposure. Normalized activity is expressed as a ratio of events/second to untreated controls (*=p of at least <0.05). FIG. 5C shows a Western blot of SNAP-25 cleavage at indicated time points after BoNT/A addition.

FIG. 6 shows ESN activity. FIG. 6A shows the effect of plating density on ESN activity. FIG. 6B depicts immunocytochemistry of ESNs. FIG. 6C Patch clamp was used to evaluate spontaneous network activity.

FIG. 7 shows the effect of clostridial neurotoxins on ESNs. FIG. 7A represents the SNARE protein cleavage following neurotoxin intoxication. The protein levels were analyzed by Western blotting. FIG. 7B depicts the immunocytochemistry of ESNs exposed to neurotoxins.

FIG. 8 shows the effects of 20 hour treatment of ESNs with botulinum neurotoxin. FIG. 8A is a representative current-clamp recording of spontaneous action potentials and excitatory post-synaptic potentials in untreated vs. 24 hour BoNT/A treated DIV 23+ESN cultures. The right panel shows quantification of spontaneous events in untreated vs. 24 hour BoNT/A treated ESNs. FIG. 8B Shows the effect of treatment of ESNs with BoNT/A (800 U/mL) for 24 hours. The left panel shows neuronal properties while the right panel shows the negative resting membrane potential. FIG. 8C Depicts the dose response of BoNT/A (0.2-800 U/mL) after 24 hours of treatment as assessed by mEPSC frequency. The top panel is representative voltage-clamp trace segments, while the bottom panel is quantification of mEPSC frequency. FIG. 8D Shows the effect of treatment of ESNs as visualized by gel mobility shifts. The top panel is a representative Western blot while the bottom panel is quantification.

FIG. 9 shows the effects of intoxication of DIV 23+ESNs with BoNT/B (FIG. 9A) or TeNT (FIG. 9B). FIG. 9C and FIG. 9D show representative traces and average mEPSC frequencies measured by MISA at 20 h after addition of BoNT/B (FIG. 9C) or TeNT (FIG. 9D).

FIG. 10 shows that DIV24⁺ ESNs functionally express iGluRs and GABARs, form glutamatergic and GABAergic synapses, and display an excitatory/inhibitory network balance. FIG. 10A shows a confocal image of DIV 24⁺ ESNs illustrating the presence of synapses (synapsin, white) at regions of axodendritic interface (Tau, blue; MAP2, magenta). FIG. 10B shows a representative voltage-clamp (−70 mV) recording showing agonist-induced currents from iGluR (left panel; glutamate, 100 μM), GABAR_{A} (middle panel; muscimol, 100 μM), and GABAR_{B} (right panel; baclofen, 100 μM) activation (n=4 for each). FIG. 10C shows continuous −70 mV voltage-clamp recordings reveal spontaneous mEPSCs and mIPSCs (left, n>8). A scaled overlay of mEPSCs and mIPSCs (right) illustrates the differential kinetics between excitatory vs. inhibitory events. Greater than 25,000 excitatory events and over 1,500 inhibitory events were analyzed. FIG. 10D shows current-clamp recordings showing the effect of GABAR antagonism (middle panel; bicuculline, 10 μM) and GABAR agonism (right panel; GABA, 100 μM) on spontaneous network communication. FIG. 10E shows representative wide field (top) and zoomed (bottom) images illustrating that ESNs contain both synapsin-positive vGluT2 (excitatory; 76.3%±4.4, SD) and synapsin-positive GAD1 (inhibitory; 23.7%±4.5, SD) synapses. Quantification of synapse distribution is presented from averaged counts of over 2,000 synapses (n=8), both distributions passed the Shapiro-Wilk normality test (a<0.05) and were significantly different from a theoretical mean of 50% (dashed line). *** indicates a P<0.001.

FIG. 11 shows that BoNT/A intoxication of ESNs decreases synaptically-mediated spontaneous activity. FIG. 11A shows representative segments of continuous current-clamp recordings of vehicle (top) vs. 24 hour BoNT/A (bottom; 20 pM) treated DIV 24⁺ ESN cultures illustrates the near complete loss of spontaneous activity (n=15). Quantification of spontaneous EPSPs and APs in untreated vs. 24 hour BoNT/A (20 pM) treated cultures (n=15) shows a significant decrease in spontaneous activity. *** indicates a P<0.001. FIG. 11B shows elicited APs from depolarizing current injection (80-120 pA) in vehicle vs. BoNT/A treated cultures illustrate that BoNT/A-intoxicated ESNs still maintain the ability to fire repetitive APs. FIG. 11C shows resting membrane potential of vehicle vs. BoNT/A treated cultures (20 pM, 24 hours, n=15) shows no change in the ability of BoNT/A-intoxicated ESNs to maintain a negative resting membrane potential.

FIG. 12 shows that BoNT/A addition results in the proteomic markers of intoxication and causes a biphasic change in mEPSC frequency. FIG. 12A shows gel-mobility shifts of total SNAP-25 illustrate the time-course of cSNAP-25 (left panel) and quantification (right panel) of cSNAP-25 as assessed by Western blot (n=4). FIG. 12 B shows immunofluorescent detection of cSNAP-25 following of BoNT/A addition (n=4) using a cleavage-specific SNAP-25 antibody (white; left panels). Zoomed images (right panels) show co-localization of cSNAP-25 (white) with synapsin (red). FIG. 12C shows representative voltage-clamp recordings at −70 mV demonstrate alterations of mEPSC frequency following BoNT/A (20 pM) addition (left). Quantification of mEPSC frequency shows an early increase in frequency later followed by a decline in synaptic events (n=24 for controls, n=8 for each time point). * indicates a P<0.05; *** indicates a P<0.001.

FIG. 13 shows that inhibitory synapses are intoxicated prior to excitatory synapses following BoNT/A addition. FIG. 13A shows representative −70 mV voltage-clamp recordings (left panel) demonstrate that inhibition of GABAR_{A} activity (bicuculline, 10 μM) and GABAR_{B} activity (CGP 55845, 1 μM) increases isolated mEPSC frequency over recordings performed in a complex background with intact inhibitory signaling. Quantification of complex mEPSCs frequency vs. isolated mEPSCs frequency (right panel, n=12 for each treatment). FIG. 13B shows representative −70 mV voltage-clamp recordings (top) illustrate the time course of isolated mEPSC and isolated mIPSC frequency following BoNT/A addition (20 pM). Quantification of isolated mEPSC vs. isolated mIPSC frequency at indicated time points following BoNT/A addition shows an accelerated decrease in mIPSCs (n=8 at each time point). FIG. 13C shows immunofluorescent detection of cSNAP-25 (white), GAD1 (red), and vGluT2 (green) reveals the preferential co-localization of cSNAP-25 with GAD1⁺ synapses after BoNT/A addition (20 pM). Quantification of the fraction of GAD1⁺ vs vGluT2⁺ synapses co-localized with cSNAP-25 at a given time-point after BoNT/A addition (n=4 coverslips per timepoint, at least 500 synapses counted per timepoint). * indicates a P<0.05; ** indicates a P<0.01.

FIG. 14 shows that epipleptiform bursting activity precedes network silencing following BoNT/A intoxication of DIV 24⁺ ESNs. FIG. 14A shows long-term current clamp recordings show the effect of vehicle or BoNT/A (20 pM) superfusion on spontaneous AP bursting behavior (n=7 for vehicle, n=5 for BoNT/A treated). Period of superfusion indicated by a bar. FIG. 14B shows quantification of network activity following superfusion with control solutions or BoNT/A shows a significant increased inter-burst interval. Time period during which superfusion occurred is indicated by a bar. Data are normalized to internal pre-superfusion baselines (−5-0 min). For determination of significance, data are compared against equivalent time windows across treatment groups. FIG. 14C shows zoomed insets from traces presented above illustrate the change in AP bursting patterns before and after control (left panel) or BoNT/A addition (right panel). FIG. 14D shows quantification of AP bursting behavior shows that BoNT/A addition significantly increases the number of APs per burst. Time period during which superfusion occurred is indicated by a bar. Data are normalized to internal pre-superfusion baselines (−5-0 min). For determination of significance, data are compared against equivalent time windows across treatment groups. * indicates a P<0.05; ** indicates a P<0.01;*** indicates a P<0.001.

### DESCRIPTION OF THE PREFERRED EMBODIMENTS

In accordance with the present invention, compositions and methods for detecting toxin or neurotoxin in a sample have been discovered. In particular, ex vivo networked neuron populations are used to detect toxins in a highly sensitive and rapid manner. Alterations in synaptic activity of networked neurons are measured to detect and identify toxins. Therefore, the compositions and methods described herein are useful for toxin detection that requires sensitivity, specificity, use with complex samples, detection of toxin activity, ease of use, and cost effectiveness. Furthermore, the compositions and methods described herein reduce the need for animal-based toxicity studies, yet serve to analyze multiple toxin functions.

### I. Compositions

Compositions of the present invention include networked neuron compositions that are susceptible to toxin intoxication to allow assaying of specific toxins. The compositions disclosed herein are useful to conduct methods that can detect femtomolar amounts of toxin in a sample.

The networked neuron compositions are isolated and include neurons derived from stem cells. In some aspects neurons are derived from stem cells in a suspended culture. In other aspects neurons are derived from stem cells in adherent cultures, such as on gelatin or in the presence of mouse embryonic fibroblasts. The neurons may be derived from any stem cell capable of differentiating into a neuron. Suitable stem cells may be totipotent cells, pluripotent cells, or multipotent cells. The stem cells may be embryonic stem cells, adult stem cells, induced pluripotent stem cells, or other stem cell known in the art or yet to be discovered. The stem cell source may be any source known in the art or yet to be discovered. Suitable stem cell sources include those from which a population of networked neurons may be derived or isolated. Examples of such stem cell sources include, without limitation, mammals, mammalian tissue, mammalian cells, primates, primate tissue, primate cells, rodents, rodent tissue, rodent cells, and any other source known in the art.

The networked neuron compositions include a population of neurons. In some aspects, the population of neurons includes at least two neurons. In some aspects, the population of neurons includes more than two neurons. Suitable neuron types that may be included in the population, without limitation, include cortical neurons, hippocampal neurons, cerebellar neurons, basal ganglia neurons, spinal cord neurons, and any type of neuron known in the art, and any combination thereof.

The networked neuron compositions include neurons that are capable of network synaptic activity. Such network synaptic activity provides the same pathophysiology of synaptic inhibition that is responsible for clinical intoxication. Networked neurons have formed active excitatory synapses, undergo trans-synaptic signaling and produce action potentials (AP), which result in networks. Such networks can exhibit a wide array of activity. Healthy networks will exhibit a balance between excitatory and inhibitory synaptic activity, such that AP propagation is stochastic (e.g., the mean and variance in AP rates are essentially equal). APs may come in bursts, or in singlets. Simultaneous observation of multiple neurons in the population may show independent events, or exhibit evidence of entrained behaviors (e.g., the observed neurons directly or indirectly communicate with one another). Overstimulation of the network results in seizurogenic behavior, such as epileptiform bursting characterized by paroxysmal depolarizing shifts. In contrast, non-networked neurons do not have formed active synapses and are unable to communicate with another neuron.

The networked neuron compositions of the invention may be genetically modified. Such genetic modification may include adding, deleting, enhancing, or silencing at least one specific gene or protein or a combination thereof. The genetic modification may be present in the stem cell before derivation or introduced after derivation into neurons. As such, the genetic modification may be introduced stably or transiently. The introduction of genetic modifications using cultured stem cells and derived cell types, such as neurons, is well established in the art.

Aspects of the present invention include stem cell derived neurons and networked neuron populations thereof that are susceptible to toxin intoxication. In some aspects, the stem cell derived neurons or networked neuron populations thereof are susceptible to toxin intoxication by about 700 pM or less, about 500 pM or less, about 400 pM or less, about 300 pM or less, about 200 pM or less, or about 100 pM or less of a toxin. In other aspects, the stem cell derived neurons or networked neuron populations thereof are susceptible to toxin intoxication by about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or networked neuron populations thereof are susceptible to toxin intoxication by about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or networked neuron populations thereof are susceptible to toxin intoxication by about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, the stem cell derived neurons or networked neuron populations thereof are susceptible to toxin intoxication by about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less pM of a toxin.

In other aspects, such neurons are susceptible to toxin intoxication from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin

Aspects of the present invention include stem cell derived neurons and networked neuron populations thereof that are susceptible to toxin intoxication. In some aspects, the stem cell derived neurons or networked neuron populations thereof are susceptible to toxin intoxication by about 700 fM or less, about 500 fM or less, about 400 fM or less, about 300 fM or less, about 200 fM or less, or about 100 fM or less of a toxin. In other aspects, the stem cell derived neurons or networked neuron populations thereof are susceptible to toxin intoxication by about 90 fM or less, about 80 fM or less, about 70 fM or less, about 60 fM or less, about 50 fM or less, about 40 fM or less, about 30 fM or less, about 20 fM or less, or about 10 fM or less of a toxin. In still other aspects, the stem cell derived neurons or networked neuron populations thereof are susceptible to toxin intoxication by about 9 fM or less, about 8 fM or less, about 7 fM or less, about 6 fM or less, about 5 fM or less, about 4 fM or less, about 3 fM or less, about 2 fM or less, or about 1 fM or less of a toxin. In yet other aspects, the stem cell derived neurons or networked neuron populations thereof are susceptible to toxin intoxication by about 0.9 fM or less, 0.8 fM or less, 0.7 fM or less, 0.6 fM or less, 0.5 fM or less, 0.4 fM or less, 0.3 fM or less, 0.2 fM or less, or about 0.1 fM or less of a toxin. In other aspects, the stem cell derived neurons or networked neuron populations thereof are susceptible to toxin intoxication by about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less fM of a toxin.

In other aspects, such neurons are susceptible to toxin intoxication from about 0.01 fM to about 100 fM, about 0.01 fM to about 75 fM, about 0.01 fM to about 50 fM, about 0.01 fM to about 25 fM, about 0.01 fM to about 20 fM, about 0.01 fM to about 15 fM, about 0.01 fM to about 10 fM, about 0.01 fM to about 5 fM, about 0.001 fM to about 100 fM, about 0.001 to about 75 fM, about 0.001 to about 50 fM, about 0.001 to about 25 fM, about 0.001 to about 20 fM, about 0.001 to about 15 fM, about 0.001 to about 10 fM, or about 0.001 to about 5 fM of toxin.

In other embodiments, the stem cell derived neurons or networked neuron populations thereof are able to uptake a toxin. Such neurons can uptake about 700 pM or less, about 500 pM or less, about 400 pM or less, about 300 pM or less, about 200 pM or less, or about 100 pM or less of a toxin. In other aspects, the stem cell derived neurons or neuron populations thereof can uptake about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or neuron populations thereof can uptake about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or neuron populations thereof can uptake about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, such neurons can uptake toxin from about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less pM of a toxin. In other aspects, such neurons can uptake toxin from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin.

In other embodiments, the stem cell derived neurons or networked neuron populations thereof are able to uptake a toxin. Such neurons can uptake about 700 fM or less, about 500 fM or less, about 400 fM or less, about 300 fM or less, about 200 fM or less, or about 100 fM or less of a toxin. In other aspects, the stem cell derived neurons or neuron populations thereof can uptake about 90 fM or less, about 80 fM or less, about 70 fM or less, about 60 fM or less, about 50 fM or less, about 40 fM or less, about 30 fM or less, about 20 fM or less, or about 10 fM or less of a toxin. In still other aspects, the stem cell derived neurons or neuron populations thereof can uptake about 9 fM or less, about 8 fM or less, about 7 fM or less, about 6 fM or less, about 5 fM or less, about 4 fM or less, about 3 fM or less, about 2 fM or less, or about 1 fM or less of a toxin. In yet other aspects, the stem cell derived neurons or neuron populations thereof can uptake about 0.9 fM or less, 0.8 fM or less, 0.7 fM or less, 0.6 fM or less, 0.5 fM or less, 0.4 fM or less, 0.3 fM or less, 0.2 fM or less, or about 0.1 fM or less of a toxin. In other aspects, such neurons can uptake toxin from about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less fM of a toxin. In other aspects, such neurons can uptake toxin from about 0.01 fM to about 100 fM, about 0.01 fM to about 75 fM, about 0.01 fM to about 50 fM, about 0.01 fM to about 25 fM, about 0.01 fM to about 20 fM, about 0.01 fM to about 15 fM, about 0.01 fM to about 10 fM, about 0.01 fM to about 5 fM, about 0.001 fM to about 100 fM, about 0.001 to about 75 fM, about 0.001 to about 50 fM, about 0.001 to about 25 fM, about 0.001 to about 20 fM, about 0.001 to about 15 fM, about 0.001 to about 10 fM, or about 0.001 to about 5 fM of toxin.

In other embodiments, the stem cell derived neurons or networked neuron populations thereof are not susceptible to a toxin. Such neurons are not susceptible to a toxin at about 700 pM or less, about 500 pM or less, about 400 pM or less, about 300 pM or less, about 200 pM or less, or about 100 pM or less of a toxin. In other aspects, the stem cell derived neurons or neuron populations thereof are not susceptible to a toxin at about 90 pM or less, about 80 pM or less, about 70 pM or less, about 60 pM or less, about 50 pM or less, about 40 pM or less, about 30 pM or less, about 20 pM or less, or about 10 pM or less of a toxin. In still other aspects, the stem cell derived neurons or neuron populations thereof are not susceptible to a toxin at about 9 pM or less, about 8 pM or less, about 7 pM or less, about 6 pM or less, about 5 pM or less, about 4 pM or less, about 3 pM or less, about 2 pM or less, or about 1 pM or less of a toxin. In yet other aspects, the stem cell derived neurons or neuron populations thereof are not susceptible to a toxin at about 0.9 pM or less, 0.8 pM or less, 0.7 pM or less, 0.6 pM or less, 0.5 pM or less, 0.4 pM or less, 0.3 pM or less, 0.2 pM or less, or about 0.1 pM or less of a toxin. In other aspects, such neurons are not susceptible to a toxin from about 0.01 pM to about 100 pM, about 0.01 pM to about 75 pM, about 0.01 pM to about 50 pM, about 0.01 pM to about 25 pM, about 0.01 pM to about 20 pM, about 0.01 pM to about 15 pM, about 0.01 pM to about 10 pM, about 0.01 pM to about 5 pM, about 0.001 pM to about 100 pM, about 0.001 to about 75 pM, about 0.001 to about 50 pM, about 0.001 to about 25 pM, about 0.001 to about 20 pM, about 0.001 to about 15 pM, about 0.001 to about 10 pM, or about 0.001 to about 5 pM of toxin.

In other embodiments, such neurons are not susceptible to a toxin at about 700 fM or less, about 500 fM or less, about 400 fM or less, about 300 fM or less, about 200 fM or less, or about 100 fM or less of a toxin. In other aspects, the stem cell derived neurons or neuron populations thereof can uptake about 90 fM or less, about 80 fM or less, about 70 fM or less, about 60 fM or less, about 50 fM or less, about 40 fM or less, about 30 fM or less, about 20 fM or less, or about 10 fM or less of a toxin. In still other aspects, the stem cell derived neurons or neuron populations thereof can uptake about 9 fM or less, about 8 fM or less, about 7 fM or less, about 6 fM or less, about 5 fM or less, about 4 fM or less, about 3 fM or less, about 2 fM or less, or about 1 fM or less of a toxin. In yet other aspects, the stem cell derived neurons or neuron populations thereof can uptake about 0.9 fM or less, 0.8 fM or less, 0.7 fM or less, 0.6 fM or less, 0.5 fM or less, 0.4 fM or less, 0.3 fM or less, 0.2 fM or less, or about 0.1 fM or less of a toxin. In other aspects, such neurons can uptake toxin from about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less fM of a toxin. In other aspects, such neurons can uptake toxin from about 0.01 fM to about 100 fM, about 0.01 fM to about 75 fM, about 0.01 fM to about 50 fM, about 0.01 fM to about 25 fM, about 0.01 fM to about 20 fM, about 0.01 fM to about 15 fM, about 0.01 fM to about 10 fM, about 0.01 fM to about 5 fM, about 0.001 fM to about 100 fM, about 0.001 to about 75 fM, about 0.001 to about 50 fM, about 0.001 to about 25 fM, about 0.001 to about 20 fM, about 0.001 to about 15 fM, about 0.001 to about 10 fM, or about 0.001 to about 5 fM of toxin.

The present invention contemplates any toxin capable of intoxicating the stem cell derived neurons of the invention. Such toxins may include, without limitation, indirect as well as direct toxins. Indirect toxins are those toxins that alter neurotransmitter release or uptake, such as causing a toxic level of Ca2⁺. Direct toxins include those toxins that directly act on a neuron, such as by uptake into a neuron or attaching to neuron membranes. While the invention encompasses any toxin capable of altering neuron homeostasis or normal neuron function, examples of toxins include, without limitation, Ablomin, Aconitine, Aconitum, Aconitum anthora, AETX, Agitoxin, Aldrin, Alpha-neurotoxin, Altitoxin, Anatoxin-a, Anisatin, Anthopleurin, Apamin, 2-Ethoxyethyl Acetate, Acibenzolar-S-methyl, Acrylamide, Aldicarb, Allethrin, Aluminum (cl or lactate), Amino-nicotinamide(6-), Aminopterin, Amphetamine(d-), Arsenic, Aspartame, Azacytidine(5-), Babycurus toxin 1, Batrachotoxin, Bestoxin, Birtoxin, BmKAEP, BmTx3, Botulinum toxin, Brevetoxin, Para-Bromoamphetamine, Bukatoxin, Benomyl, Benzene, Bioallethrin, Bis(tri-n-butyltin)oxide, Bisphenol A, Bromodeoxyuridine(5-), Butylated Hydroxy Anisol, Butylated hydroxytoluene, Calcicludine, Calciseptine, Carbon disulfide, Charybdotoxin, Para-Chloroamphetamine, Cicutoxin, Ciguatoxin, Clostridium botulinum, Conantokins, Conhydrine, Coniine, Conotoxin, Contryphan, Curare, Cyanide poisoning, Cylindrospermopsin, Cypermethrin, Cadmium, Caffeine, Carbamazepine, Carbaryl, Carbon monoxide, Chlordecone, Chlordiazepoxide Chlorine dioxide, Chlorpromazine, Chlorpyrifos Cocaine, Colcemid, Colchicine, Cypermethrin, Cytosine Arabinoside, Delta atracotoxin, Dendrotoxin, Dexamethasone, Diamorphine hydrochloride, Dieldrin, 5,7-Dihydroxytryptamine, Diisopropyl fluorophosphate, Dimethylmercury, Discrepin, Domoic acid, Dortoxin, DSP-4, Diazepam, DEET, Deltamethrin, Diazinon, Dieldrin, Diethylstilbestrol, Diphenylhydantoin, Epidermal Growth Factor, Ethanol, Ethylene thiourea, Falcarinol, Flourouracil(5-), Fluazinam, Fluoride, Gabaculine, Ginkgotoxin, Grammotoxin, Grayanotoxin, Griseofulvin, Hainantoxin, Hefutoxin, Helothermine, Heteroscodratoxin-1, Histrionicotoxin, Hongotoxin, Huwentoxin, Haloperiodol, Halothane, Heptachlor, Hexachlorobenzene, Hexachlorophene, Hydroxyurea, Para-lodoamphetamine, Ibotenic acid, Ikitoxin 5-Iodowillardiine, Imminodiproprionitrile (IDPN), Jingzhaotoxin, Ketamine, Kurtoxin, Latrotoxin, Alpha-Latrotoxin, Lq2, Lead, LSD, Lindane, Maitotoxin, Margatoxin, Maurotoxin, Methanol, Methiocarb, Beta-Methylamino-L-alanine, N-Methylconiine, MPP+, MPTP, Myristicin, Maneb, Medroxyprogesterone, Mepivacaine, Methadone, Methanol, Methimazole, Methylparathion, Monosodium Glutamate, MPTP, Naloxone, Nemertelline, Neosaxitoxin, Nicotine, Naltrexone, 2-Methoxyethanol, Methylazoxymethanol, Methylmercury, Oxidopamine, Ozone, Oenanthotoxin, Oxalyldiaminopropionic acid, Palytoxin, Penitrem A, Phaiodotoxin, Phenol, Phoneutria nigriventer toxin-3, Phrixotoxin, Polyacrylamide, Poneratoxin, Psalmotoxin, Pumiliotoxin, Paraquat, Parathion (ethyl), PBDEs, PCBs (generic), Penicillamine, Permethrin, Phenylacetate Phenylalanine (d,l), di-(2-ethylhexyl) Phthalate, Propylthiouracil, Raventoxin, Resiniferatoxin, Retinoids/vit.A/isotretinoin, Samandarin, Saxitoxin, Scyllatoxin, Sea anemone neurotoxin, Shiga toxin, Slotoxin, SNX-482, Stichodactyla toxin, Salicylate, Taicatoxin, Taipoxin, Tamapin, Tertiapin, Tetanospasmin, Tetanus toxin, Tetraethylammonium, Tetrodotoxin, Tityustoxin, Tricresyl phosphate, Tebuconazole, Tellurium (salts), Terbutaline, Thalidomide, THC, Toluene, Triamcinolone, Tributyltin chloride, Trichlorfon, Trichloroethylene, Triethyllead, Triethyltin, Trimethyltin, Trypan blue, Urethane, Valproate, Vanillotoxin, Veratridine, Vincristine. Further, suitable toxins may include, without limitation, clostridial neurotoxins such as those produced by the Clostridium genus of bacteria. Such clostridial neurotoxins include, without limitation, botulinum neurotoxin, butyricum neurotoxin, baratii neurotoxin, and tetanus neurotoxin. Examples of botulinum neurotoxin include serotype BoNT/A1, serotype BoNT/A2, serotype BoNT/A3, serotype BoNT/A4, serotype BoNT/A5, serotype BoNT/B1, serotype BoNT/B2, serotype BoNT/B3, serotype BoNT/B(bivalent), serotype BoNT/B (nonproteolytic), serotype BoNT/C, serotype BoNT/D, serotype BoNT/E1, serotype BoNT/E2, serotype BoNT/E3, serotype BoNT/E4, serotype BoNT/E5 serotype BoNT/E6, serotype BoNT/E7, serotype BoNT/E8, serotype BoNT/E9, serotype BoNT/F1, serotype BoNT/F2, serotype BoNT/F3, serotype BoNT/F4, serotype BoNT/F5, serotype BoNT/F6, serotype BoNT/F7, serotype BoNT/G, serotype BoNT/H and serotypes and subtypes yet to be discovered. Examples of butyricum neurotoxin include serotypes BoNT/E4, and serotype BoNT/E5. Examples of baratii neurotoxin include serotype BoNT/F and serotypes and subtypes yet to be discovered. Examples of tetanus neurotoxin include serotype TeNT and serotypes and subtypes yet to be discovered.

Preferred toxins included Botulinum toxin, Tetanus toxin, Latrotoxin, Shiga toxin, Tetrodotoxin, Conotoxin, and combinations thereof. Preferred Botulinum toxins include serotype /A, serotype /B, serotype /C, serotype /D, serotype /E, serotype /F, serotype /G, serotype /H, subtypes thereof, or combinations thereof.

### II. Methods

The present invention provides novel assays for detecting the presence or absence of an active neurotoxin. The methods disclosed herein reduce the need for animal-based toxicity studies. The methods disclosed herein may be used to analyze crude and bulk samples as well as highly purified toxins and formulated toxin products. As non-limiting examples, methods of the invention may be useful for detecting the presence or activity of a toxin in a food or beverage sample; to assay a sample from a human or animal, for example, exposed to a toxin or having one or more symptoms of toxin exposure; to follow activity during production and purification of toxin; to assay formulated toxin products such as pharmaceuticals or cosmetics; to identify the toxin in a sample; to identify or analyze neurotoxin neutralizing agents; or for other reasons that become apparent to a skilled artisan.

### Detecting Neurotoxin

The present invention includes methods of detecting neurotoxin in a sample by contacting the sample to a networked neuron composition that is capable of network synaptic activity. The network synaptic activity is measured using methods known in the art. In some aspects the network synaptic activity is measured before and after the sample has contacted the networked neuron composition. The measurements are compared and a change in network synaptic activity is correlated with neurotoxin detection. A decrease in synaptic activity is correlated with the presence of neurotoxin in the sample. An increase, or no change, in network synaptic activity is correlated with the absence of neurotoxin in the sample.

In some aspects, multiple measurements are taken before the sample has contacted the networked neuron composition. In some aspects, multiple measurements are taken after the sample has contacted the networked neuron composition. In some aspects, such multiple measurements are at the same time point. In some aspects, such multiple measurements are at different time points. In some aspects, multiple measurements are taken at the same time point and for multiple time points. Multiple measurements may include a first, second, third, fourth, fifth, sixth, seventh, eighth, ninth, tenth, eleventh, twelfth, thirteenth, fourteenth, fifteenth, and more synaptic activity measurements.

A control network synaptic activity may also be measured for comparing the network synaptic activity of the sample-contacted composition. Suitable control network synaptic activity may be the network synaptic activity measured before the sample is contacted to the networked neuron composition. In some embodiments, the control network synaptic activity may be the network synaptic activity measured in a networked neuron composition that is treated or maintained identically to the sample-contacted composition, except that a sample has not been contacted with the control composition.

In some embodiments, a decrease in networked synaptic activity is indicative of neurotoxin presence in a sample if the decrease is about 10% or more compared to the control networked synaptic activity. In other embodiments, a decrease in networked synaptic activity is indicative of neurotoxin presence in a sample if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

In some embodiments, an increase in networked synaptic activity is indicative of the absence of neurotoxin in a sample if the increase is about 10% or more compared to the control networked synaptic activity. In other embodiments, an increase in networked synaptic activity is indicative of the absence of neurotoxin in a sample if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

The methods of the invention include detecting toxin or neurotoxin in a sample with high sensitivity. The specific activity of the toxin can be described as the lethal level divided by unit mass. Specific activity is typically described in terms of the number of mouse LD50 values present in a given mass of toxin. For example, a specific activity of 1×10⁸ mouse lethal units (U) per mg indicates that a single mg of toxin contains sufficient toxin molecules to lethally intoxicate 1×10⁸ mice in the mouse lethal assay. Another way of describing the toxin is by using the concentration, which is equal to the amount of toxin divided by volume. Concentration may be expressed as mg/mL, moles/L or mouse lethal units/mL. Once the specific activity has been determined for a given toxin formulation in terms of U/mg, it can easily be converted to concentration based on the volume in which the toxin is prepared. For example, the use of 10 U/mL is a specific description that simply states that in 1 mL of an aqueous media, there is sufficient toxin to provide sufficient toxin to 10 mice to expose each to 1 LD50. According to one aspect, the invention relates to a method for determining the U/ml comprising the steps of i) determine the specific activity of the preparation, which may be expressed as a lethal activity divided by mass; and, ii) convert the specific activity to concentration based on the known volume. This allows for direct comparison among different toxin lots, sources, serotypes, subtypes, and assays based on the common activity of mouse lethality.

The level of toxin or neurotoxin in a sample may be detected at about 2.0 U/mL or less. More preferably, the toxin or neurotoxin in a sample may be detected at a level of about 1.0 U/mL or less. In some embodiments, the toxin or neurotoxin in a sample may be detected at a level of about 1.9, 1.8, 1.7, 1.6, 1.5, 1.4, 1.3, 1.2, 1.1, 1.0, 0.95, 0.9, 0.85, 0.80, 0.75, 0.70, 0.65, 0.60, 0.55, 0.50, 0.45, 0.40, 0.35, 0.30, 0.25, 0.20, 0.15, 0.10, 0.05, 0.01 U/mL, or less. In some embodiments, the toxin or neurotoxin in a sample may be detected at a level in the range of 0.001-10 U/mL, more preferably 0.001-5 U/mL, more preferably 0.001-2 U/mL, more preferably 0.001-1 U/mL, more preferably 0.001-0.5 U/mL, more preferably 0.01-0.3 U/mL, more preferably 0.01-0.25 U/mL, more preferably 0.01-0.2 U/mL, more preferably 0.01-0.15 U/mL.

In some embodiments, the concentration of toxin or neurotoxin in a sample may be detected at a level of about 700 fM or less, about 500 fM or less, about 400 fM or less, about 300 fM or less, about 200 fM or less, or about 100 fM or less of a toxin. the concentration of toxin or neurotoxin in a sample may be detected at a level of about 90 fM or less, about 80 fM or less, about 70 fM or less, about 60 fM or less, about 50 fM or less, about 40 fM or less, about 30 fM or less, about 20 fM or less, or about 10 fM or less of a toxin. the concentration of toxin or neurotoxin in a sample may be detected at a level of about 9 fM or less, about 8 fM or less, about 7 fM or less, about 6 fM or less, about 5 fM or less, about 4 fM or less, about 3 fM or less, about 2 fM or less, or about 1 fM or less of a toxin. the concentration of toxin or neurotoxin in a sample may be detected at a level of about 0.9 fM or less, 0.8 fM or less, 0.7 fM or less, 0.6 fM or less, 0.5 fM or less, 0.4 fM or less, 0.3 fM or less, 0.2 fM or less, or about 0.1 fM or less of a toxin. the concentration of toxin or neurotoxin in a sample may be detected at a level of about 900, 850, 800, 750, 700, 650, 625, 600, 575, 550, 525, 500, 475, 450, 425, 400, 375, 350, 325, 300, 275, 250, 225, 200, 175, 150, 125, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, 2, 1, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, 0.04, 0.03, 0.02, 0.01 or less fM of a toxin. the concentration of toxin or neurotoxin in a sample may be detected at a level of about 0.01 fM to about 100 fM, about 0.01 fM to about 75 fM, about 0.01 fM to about 50 fM, about 0.01 fM to about 25 fM, about 0.01 fM to about 20 fM, about 0.01 fM to about 15 fM, about 0.01 fM to about 10 fM, about 0.01 fM to about 5 fM, about 0.001 fM to about 100 fM, about 0.001 to about 75 fM, about 0.001 to about 50 fM, about 0.001 to about 25 fM, about 0.001 to about 20 fM, about 0.001 to about 15 fM, about 0.001 to about 10 fM, or about 0.001 to about 5 fM of toxin.

The methods of invention include detecting toxin or neurotoxin in a sample rapidly. The presence of a toxin or neurotoxin in a sample may be detected within about 0.5 hours to about 36 hours after the sample has contacted the networked neuron composition. In some aspects, the presence of a toxin or neurotoxin in a sample may be detected within about 0.5 hours or less. In some aspects, the presence of a toxin or neurotoxin in a sample may be detected within about 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, 30 or more hours after the sample has contacted the networked neuron composition.

### Evaluating Neurotoxin Neutralizing Agents

Another aspect includes methods of evaluating the efficacy of neurotoxin neutralizing agents. Such method includes providing a networked neuron composition and contacting the composition with a neurotoxin to form an intoxicated composition. The intoxicated composition is contacted with a neurotoxin neutralizing agent of interest. The network synaptic activity is measured by methods known in the art and described herein. The measurements taken are compared and correlated with neurotoxin neutralization. A decrease in synaptic activity is correlated with the absence of neurotoxin neutralization. Preferably, a decrease in synaptic activity measured before the neurotoxin was contacted to the networked neuron composition and in synaptic activity measured after the neurotoxin neutralization agent was added to the intoxicated composition is correlated with the absence of neurotoxin neutralization. An increase, or no change, in network synaptic activity is correlated with the presence of neurotoxin neutralization. Preferably, an increase, or no change, in network synaptic activity measured after neurotoxin is contacted to the networked neuron composition compared with that measured after the neurotoxin neutralization agent was added to the intoxicated composition is correlated with the presence of neurotoxin neutralization.

In some aspects the network synaptic activity is measured before and after the neurotoxin has contacted the networked neuron composition. In some aspects the network synaptic activity is measured before and after the neurotoxin neutralizing agent has contacted the intoxicated composition. In some aspects, multiple measurements are taken the neurotoxin neutralizing agent has contacted the intoxicated composition. In some aspects, multiple measurements are taken after the neurotoxin neutralizing agent has contacted the intoxicated composition. In some aspects, such multiple measurements are at the same time point. In some aspects, such multiple measurements are at different time points. In some aspects, multiple measurements are taken at the same time point and for multiple time points. Multiple measurements may include a first, second, third, fourth, fifth, sixth, seventh, eighth, ninth, tenth, eleventh, twelfth, thirteenth, fourteenth, fifteenth, and more synaptic activity measurements.

A control network synaptic activity may also be measured for comparing the network synaptic activity of the intoxicated composition. Suitable control network synaptic activity may be the network synaptic activity measured before the neutralizing agent is contacted to the intoxicated composition. In some embodiments, the control network synaptic activity may be the network synaptic activity measured in a networked neuron composition that is treated or maintained identically to the intoxicated composition, except that a neutralizing agent has not been contacted with the control composition.

In some embodiments, a decrease in networked synaptic activity is indicative of an absence of neurotoxin neutralization if the decrease is about 10% or more compared to the control networked synaptic activity. In other embodiments, a decrease in networked synaptic activity is indicative of an absence of neurotoxin neutralization if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

In some embodiments, an increase in networked synaptic activity is indicative of neurotoxin neutralization if the increase is about 10% or more compared to the control networked synaptic activity. In other embodiments, an increase in networked synaptic activity is indicative of neurotoxin neutralization if the increase is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

### Detecting Synaptic Preservation

Another aspect includes methods of detecting synaptic preservation. Such method includes providing a networked neuron composition and contacting the composition with a sample containing neurotoxin to form an intoxicated composition. The intoxicated composition is contacted with a neurotoxin neutralizing agent. The network synaptic activity is measured by methods known in the art and described herein. The measurements taken are compared and correlated with synaptic preservation. A decrease in synaptic activity is correlated with the absence of synaptic preservation. Preferably, a decrease in synaptic activity measured after the sample was contacted to the networked neuron composition and in synaptic activity measured after the neurotoxin neutralization agent was added to the intoxicated composition is correlated with the absence of synaptic preservation. An increase, or no change, in network synaptic activity is correlated with the presence of synaptic preservation. Preferably, an increase, or no change, in network synaptic activity measured after the sample is contacted to the networked neuron composition compared with that measured after the neurotoxin neutralization agent was added to the intoxicated composition is correlated with the presence of synaptic preservation.

In some aspects the network synaptic activity is measured before and after the sample has contacted the networked neuron composition. In some aspects the network synaptic activity is measured before and after the neurotoxin neutralizing agent has contacted the intoxicated composition. In some aspects, multiple measurements are taken before the neurotoxin neutralizing agent has contacted the intoxicated composition. In some aspects, multiple measurements are taken after the neurotoxin neutralizing agent has contacted the intoxicated composition. In some aspects, such multiple measurements are at the same time point. In some aspects, such multiple measurements are at different time points. In some aspects, multiple measurements are taken at the same time point and for multiple time points. Multiple measurements may include a first, second, third, fourth, fifth, sixth, seventh, eighth, ninth, tenth, eleventh, twelfth, thirteenth, fourteenth, fifteenth, and more synaptic activity measurements.

A control network synaptic activity may also be measured for comparing the network synaptic activity of the intoxicated composition. Suitable control network synaptic activity may be the network synaptic activity measured before the neutralizing agent is contacted to the intoxicated composition. In some embodiments, the control network synaptic activity may be the network synaptic activity measured in a networked neuron composition that is treated or maintained identically to the intoxicated composition, except that a neutralizing agent has not been contacted with the control composition.

In some embodiments, a decrease in networked synaptic activity is indicative of an absence of synaptic preservation if the decrease is about 10% or more compared to the control networked synaptic activity. In other embodiments, a decrease in networked synaptic activity is indicative of an absence of neurotoxin neutralization if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

In some embodiments, an increase in networked synaptic activity is indicative of synaptic preservation if the increase is about 10% or more compared to the control networked synaptic activity. In other embodiments, an increase in networked synaptic activity is indicative of synaptic preservation if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

### Identification of Neurotoxins

Another aspect includes methods of identifying the neurotoxin in a sample. Such method includes providing a networked neuron composition and contacting the composition with a sample exposed to neurotoxin to form an intoxicated composition. The intoxicated composition is contacted with a neurotoxin neutralizing agent of known specificity. The network synaptic activity is measured by methods known in the art and described herein. The measurements taken are compared and correlated with neurotoxin identity. A decrease in synaptic activity is correlated with the absence of neutralizing agent specificity. Preferably, a decrease in synaptic activity measured before the neurotoxin was contacted to the networked neuron composition and in synaptic activity measured after the neurotoxin neutralization agent was added to the intoxicated composition is correlated with the absence of neutralizing agent specificity. An increase, or no change, in network synaptic activity is correlated with the presence of neutralizing agent specificity. Preferably, an increase, or no change, in network synaptic activity measured after neurotoxin is contacted to the networked neuron composition compared with that measured after the neurotoxin neutralization agent was added to the intoxicated composition is correlated with the presence of neutralizing agent specificity.

The neurotoxin identity may be inferred from the specificity of the neutralizing agent. If the neutralizing agent has specificity to more than one neurotoxin, a combination of neutralizing agents may be used in separate trials with this method to determine the identity of the neurotoxin.

In some aspects the network synaptic activity is measured before and after the neurotoxin has contacted the networked neuron composition. In some aspects the network synaptic activity is measured before and after the neurotoxin neutralizing agent has contacted the intoxicated composition. In some aspects, multiple measurements are taken the neurotoxin neutralizing agent has contacted the intoxicated composition. In some aspects, multiple measurements are taken after the neurotoxin neutralizing agent has contacted the intoxicated composition. In some aspects, such multiple measurements are at the same time point. In some aspects, such multiple measurements are at different time points. In some aspects, multiple measurements are taken at the same time point and for multiple time points. Multiple measurements may include a first, second, third, fourth, fifth, sixth, seventh, eighth, ninth, tenth, eleventh, twelfth, thirteenth, fourteenth, fifteenth, and more synaptic activity measurements.

A control network synaptic activity may also be measured for comparing the network synaptic activity of the intoxicated composition. Suitable control network synaptic activity may be the network synaptic activity measured before the neutralizing agent is contacted to the intoxicated composition. In some embodiments, the control network synaptic activity may be the network synaptic activity measured in a networked neuron composition that is treated or maintained identically to the intoxicated composition, except that a neutralizing agent has not been contacted with the control composition.

In some embodiments, a decrease in networked synaptic activity is indicative of an absence of neurotoxin neutralization if the decrease is about 10% or more compared to the control networked synaptic activity. In other embodiments, a decrease in networked synaptic activity is indicative of an absence of neurotoxin neutralization if the decrease is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

In some embodiments, an increase in networked synaptic activity is indicative of neurotoxin neutralization if the increase is about 10% or more compared to the control networked synaptic activity. In other embodiments, an increase in networked synaptic activity is indicative of neurotoxin neutralization if the increase is about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100% or more compared to the control networked synaptic activity.

### Measuring Network Synaptic Activity

Methods of the present invention include measuring synaptic activity or network synaptic activity. Suitable methods of measuring such synaptic activity include methods known in the art. Such methods include, without limitation, whole-cell patch clamp electrophysiology, extracellular electrophysiology, amperometry, multi-electrode arrays, neurotransmitter release assays (i.e. radioactive labeled, fluorescent, enzymatic), live cell imaging with label indicators (i.e. genetically encoded indicators, GcaMPs, etc.; permeant dyes, Fluo4, etc.), genetic reporters of activity-dependent genes (i.e. arc, jun, fos, etc.), any methods known in the art or yet to be discovered, and combinations thereof.

The methods of the invention include measuring the network synaptic activity between at least two neurons that have formed a synapse. In some embodiments, the network synaptic activity is measured between 2 neurons at a time. In other embodiments, the network synaptic activity is measured between more than neurons and includes the global effects of synaptic firing among a population of neurons.

It is envisioned that a wide variety of processing formats may be used in conjunction with the methods disclosed herein, including, without limitation, manual processing, partial automated-processing, semi-automated-processing, full automated-processing, high throughput processing, high content processing, and the like or any combination thereof.

### III. Kits

The present invention provides articles of manufacture and kits containing materials useful for the methods described herein. The article of manufacture may include a container of a composition as described herein with a label. Suitable containers include, for example, bottles, vials, and test tubes. The containers may be formed from a variety of materials such as glass or plastic. The container holds a composition which is useful for detecting toxins, for example, in samples suspected of containing toxins. In some embodiments, the container holds a composition which is useful for identifying toxins. In some embodiments, the container holds a composition which is useful for evaluating toxin neutralizing agents. The composition includes stem cell derived neurons capable of network synaptic activity and may further include culture media for maintaining such neurons. The label on the container may indicate that the composition is useful for detecting toxins and may also indicate directions for detection. In some embodiments, the label on the container may indicate that the composition is useful for identifying toxins and may also indicate directions for identification. In some embodiments, the label on the container may indicate that the composition is useful for evaluating toxin neutralizing agents and may also indicate directions for evaluation.

### DEFINITIONS

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

As used herein, the term “about” when qualifying a value of a stated item, number, percentage, or term refers to a range of plus or minus ten percent of the value of the stated item, percentage, parameter, or term.

The term “ex vivo”, as used herein, refers to a population of cells maintained outside of an organism.

The term “isolated”, as used herein, refers to a biological substance, however constructed, synthesized, or derived, which is locationally distinct from its natural location. The definition includes the isolated biological substance in all its forms other than the natural state. For example, the isolated biological substance may be located in a vial, container, defined-culture media, solution, or a different organism from which it originated. Such biological substances may include, without limitation, tissue, a cell, population of cells, population of tissues, DNA, RNA, proteins, peptides, cell organelles, and any other biological substance known in the art.

The phrase “network synaptic activity”, as used herein, means the synaptic activity occurring between 2 or more neurons that have formed synapses. Such synaptic activity includes the communication between 2 neurons, as well as the activity occurring among multiple neurons driven by the global effects of synaptic firing.

The term “neurotoxin”, as used herein, refers to any toxin capable of intoxicating neurons, or inhibiting normal neuron function. Such toxins may include, without limitation, indirect as well as direct toxins. Indirect toxins are those toxins that alter neurotransmitter release or uptake, such as causing a toxic level of Ca2⁺. Direct toxins include those toxins that directly act on a neuron, such as by uptake into a neuron or attaching to neuron membranes.

The term “neurotoxin neutralization”, as used herein, refers to the restoration of neurotoxin effected synaptic activity or cessation in the decline of neurotoxin effected synaptic activity.

The term “neutralization agent”, as used herein, refers to an agent that restores neurotoxin effected synaptic activity or causes a cessation in the decline of neurotoxin effected synaptic activity. Suitable neutralization agents include antibodies, molecules, small molecules, chemical moieties, peptides, proteins, pharmaceutical formulations, pharmaceutical compositions, and other agents known in the art or yet to be discovered that are capable of restoring synaptic activity after neuron intoxication or ceasing the decline in synaptic activity after neuron intoxication.

The term “neutralization agent specificity”, as used herein, refers to the neurotoxin the neutralization agent is effective against. For instance, a neutralization agent may neutralize BoNT/A toxicity effects, but not neutralize the effects of other neurotoxins. The neutralization agent specificity for such neutralization agent would be BoNT/A specificity. Neutralization agents may have specificity for one or more neurotoxins. For instance, a neutralization agent may neutralize the effects of 1, 2, 3, 4, 5, 6, 7, 8, 9, 10, 11, 12, 13, 14, 15, 16, 17, 18, 19, 20 or more neurotoxins.

As used herein, the term “sample” refers any composition that contains or potentially contains a toxin or neurotoxin. A variety of samples may be used with the methods disclosed herein including, without limitation, purified, partially purified, or unpurified toxin; toxin with naturally or non-naturally occurring sequence; recombinant toxin; chimeric toxin containing structural elements from multiple toxin species or subtypes; bulk toxin; formulated toxin product; foods; cells or crude, fractionated or partially purified cell lysates, for example, engineered to include a recombinant nucleic acid encoding a toxin or gene of interest; bacterial, baculoviral and yeast lysates; raw, cooked, partially cooked or processed foods; beverages; animal feed; soil samples; water samples; pond sediments; lotions; cosmetics; and clinical formulations. The term “sample” also encompasses tissue samples, including, without limitation, mammalian tissue samples, livestock tissue samples such as sheep, cow and pig tissue samples; primate tissue samples; and human tissue samples. Such samples encompass, without limitation, intestinal samples, saliva, excretions, feces, urine, blood, samples from wounds, and mucous. It is also contemplated that the term “sample” also encompasses those of a specific environment. Such samples may include soil, water, biomass, plant, tree, air, gas, or combinations thereof.

The term “stem cell derived” refers to a population of cells that are the result of induced stem cell differentiation. For example, stem cell derived neurons are a population of neurons resulting from exogenously providing differentiation factors to a stem cell or population of stem cells to promote differentiation into neurons.

The term “subject”, as used herein, refers to a living organism having a central nervous system. In particular, subjects include, but are not limited to, human subjects or patients and companion animals. Exemplary companion animals may include domesticated mammals (e.g., dogs, cats, horses), mammals with significant commercial value (e.g., dairy cows, beef cattle, sporting animals), mammals with significant scientific values (e.g., captive or free specimens of endangered species), or mammals which otherwise have value. Suitable subjects also include: mice, rats, dogs, cats, ungulates such as cattle, swine, sheep, horses, and goats, lagomorphs such as rabbits and hares, other rodents, and primates such as monkeys, chimps, and apes. Subjects may be of any age including new born, adolescence, adult, middle age, or elderly.

The term “synaptic preservation”, as used herein, refers to the cessation of synaptic activity decline or detrimental effects on synaptic activity.

As used herein, the terms “toxin” and “neurotoxin”, as used herein, are interchangeable and refer to any substance, molecule, or composition that is capable of intoxicating neurons. Such toxins may include, without limitation, indirect as well as direct toxins. Indirect toxins are those toxins that alter neurotransmitter release or uptake, such as causing a toxic level of Ca2+. Direct toxins include those toxins that directly act on a neuron, such as by uptake into a neuron or attaching to neuron membranes.

### EXAMPLES

The following examples are simply intended to further illustrate and explain the present invention. The invention, therefore, should not be limited to any of the details in these examples.

### Example 1

### Materials and Methods

ESN Culture and BoNT/A Intoxication.

R1 murine embryonic stem cells were maintained and differentiated into ESNs as previously described in McNutt P. et al. (2011) Biochemical and Biophysical Research communications 405(1):85-90. Experiments were conducted on ESNs at DIV 24-34 (DIV 24+). Throughout the study, data were collected from 10 independent ESN differentiations over 6 months, with no apparent change in functional response to intoxication.

Independent ESN cultures were checked for synaptic functionality using whole-cell patch clamp recordings to ensure complex mEPSC frequency exceeded 4.0 Hz prior to experimentation. Ten out of 11 differentiations passed this criteria and were included in this study. BoNT/A1 (specific activity=2.5×10⁸ U; Metabiologics, Madison, Wis.) was diluted in extracellular recording buffer and added directly to DIV 24⁺ ESNs at a concentration of 20 pM for the indicated amount of time.

Electrophysiology.

Whole-cell patch clamp recordings were performed as previously described in Gut I. M. et al. (2013) PloS One 8(5):e64423. Briefly, for mEPSC detection and current-clamp recordings, recording pipettes were filled with an intracellular recording buffer containing (in mM): 140 K-gluconate, 5 NaCl, 2 Mg-ATP, 0.5 Li-GTP, 0.1 CaCl₂, 1 MgCl₂, 1 ethylene glycol-bis (b-aminoethyl ether)-N,N,N,N-tetraacetic acid (EGTA) and 10 HEPES. For mIPSC detection, recording pipettes were filled with an intracellular recording buffer containing (in mM): 140 CsCl, 5 NaCl, 2 Mg-ATP, 0.5 Li-GTP, 0.1 CaCl₂, 1 MgCl₂, 1 EGTA, and 10 HEPES. Cultures were bathed in an extracellular recording buffer (ERB) containing (in mM): 140 NaCl, 3.5 KCl, 1.25 NaH₂PO₄, 2 CaCl₂, 1 MgCl₂, 10 Glucose, 10 HEPES. All buffers were adjusted to pH of 7.3 with NaOH or KOH and an osmolarity of 315±10 mOsm with glucose prior to recording. For mEPSC and mIPSC recordings, ERB was supplemented with 5 pM tetrodotoxin. For isolated mEPSCs, 10 pM bicuculline and 1 pM CGP 55845 were added to ERB. For isolated mIPSCs, 50 pM APV and 10 pM CNQX were added to ERB. For current-clamp recordings, spontaneous EPSPs and APs were detected using MiniAnalysis (Synaptosoft, Inc., Decatur, Ga.) with the following settings: threshold, 5 mV; period to search for local max, 10,000 μs; time before peak, 10,000 μs; period for decay, 4000 μs; fraction of peak to decay, 0.37; period to average baseline, 1000 μs; area threshold, 50; number of points to average peak, 1; direction, positive. For AP bursting quantification, software recommended AP detection parameters were used with the following settings: threshold, 25 mV; period to search for local max, 10,000 μs; time before peak, 10,000 μs; period for decay, 4000 μs; fraction of peak to decay, 0.37; period to average baseline, 1000 μs; area threshold, 50; number of points to average peak, 1; direction, positive. Secondary burst analysis was performed with the following settings: minimum number of consecutive events to be considered a burst, 2; maximum interevent interval between two events, 1 s. A 10 minute baseline recording was captured, with the 5 minutes immediately preceding superfusion serving as control values. For mEPSC and mIPSC detection, ESNs were voltage-clamped to −70 mV and recorded continuously for at least 4 minutes. mEPSCs and mIPSCs events were detected with MiniAnalysis using software recommended mEPSC and mIPSC detection parameters. To account for the increased background noise during mIPSC recordings, an amplitude of 7.5 times the background signal was used as a threshold for event detection of each recording. For agonist-induced iGluR and GABAR currents, neurons were perfused using a three barrel Fast Step system (Warner Instruments, Hamden, Conn.). For long-term current clamp recordings, vehicle or BoNT/A was gradually perfused into ERB using gravity-fed plastic tubing over the course of 3 to 4 minutes until final concentration was reached.

Immunoassays.

Protein from ESN cultures for Western blot detection was harvested and separated by SDS-PAG. Antibodies used for Western blotting in this study include SNAP-25 (Covance, Princeton, N.J.) and syntaxin (Synaptic Systems, Gottingen, Germany). For immunocytochemistry, ESNs on 18-mm coverslips were fixed in 4% paraformaldehyde, stained, and visualized by microscopy. Antibodies used in this study include anti-Tau (Synaptic Systems), anti-MAP2 (Synaptic Systems), anti-SNAP-25 (Covance), anti-cleavage-specific SNAP-25 (Research and Diagnostic Antibodies, Las Vegas, Nev.), anti-synapsin (Synaptic Systems), antivGluT2 (Synaptic Systems), anti-gad1/gad67 (Synaptic Systems).

Statistical Analysis.

Statistical significance among means was determined using one-way ANOVA testing, and P values were calculated against controls with the Dunnett's post-hoc test. Statistical significance between grouped means was determined using two-way ANOVA testing, and P values were calculated between means with Bonferroni's post-hoc test. For binary comparisons of means, the Student's t-test was used. For quantification of glutamatergic vs. GABAergic synapses, a Shapiro-Wilk normality test was first performed to confirm that data were normally distributed followed by a one sample t-test comparing each population against a theoretical mean of 50%. Unless otherwise stated, all quantitative data are presented as mean±the standard error of the mean, with the following markers of statistical significance: * indicates a P<0.05; ** indicates a P<0.01; *** indicates a P<0.001.

### Example 2

### ESNs Develop a Complex Network of Synaptic Connections

To determine if ESNs could be used to develop an assay model that includes all pathophysiological steps involved in toxin activity, a population of ESNs were analyzed. ESNs were differentiated from suspension-cultured mouse ESCs as previously described (Hubbard et al. 2012, BMC Neurosci 13:127). For whole cell electrophysiology recordings, 5-7 MΩ pipettes were pulled from capillary glass and filled with an intracellular recording buffer containing (in mM): 140 K-gluconate, 5 NaCl, 2 Mg-ATP, 0.5 Li-GTP, 0.1 CaCl₂, 1 MgCl₂, 1 EGTA and 10 HEPES. Cultures were bathed in an extracellular recording buffer containing (in mM): 140 NaCl, 3.5 KCl, 1.25 NaH₂PO₄, 2 CaCl₂, 1 MgCl₂, 10 Glucose, and 10 HEPES. The osmolarity and pH of both solutions was checked prior to recording. Data was acquired at 20-22° C. with a HEKA EPC10 amplifier and Heka Patchmaster 2.53 software. Data analysis and graphing was performed in Heka Fitmaster 2.53, Igor Pro v6, and Prism v6. Data was adjusted post-hoc for a liquid junction potential of −15 mV. Continuous recordings of spontaneous activity were collected for 2-4 minutes/neuron. Continuous readings from neurons that were unable to fire elicited action potentials or display voltage-gated currents after recording were discarded. Analysis of spontaneous events was performed using MiniAnalysis v6 with the following settings: threshold, 5 mV; period to search for local max, 10,000 ms; time before peak, 10,000 ms; period for decay, 4000 ms; fraction of peak to decay, 0.37; period to average baseline, 1000 ms; area threshold, 50; number of points to average peak, 1. Immunocytochemistry and western blotting were performed using standard techniques.

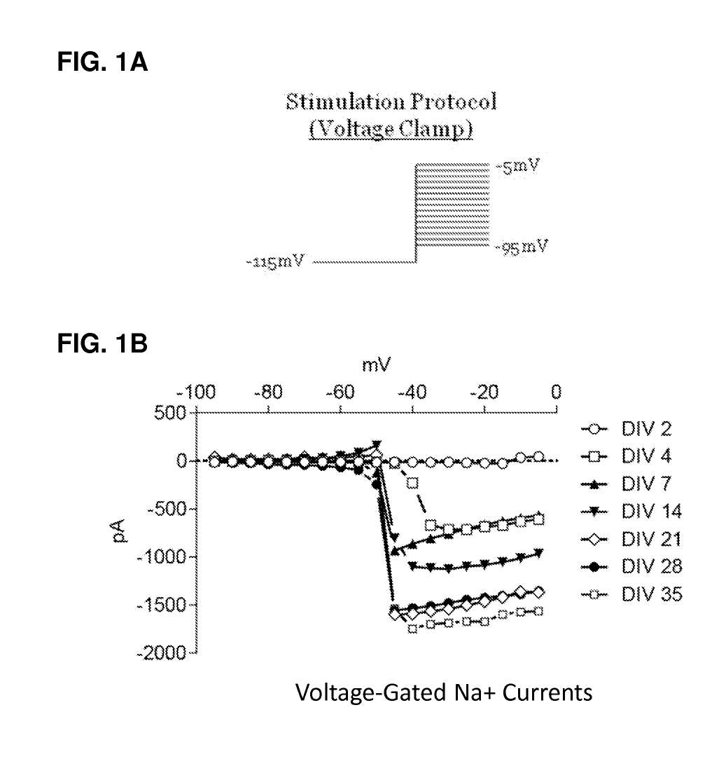

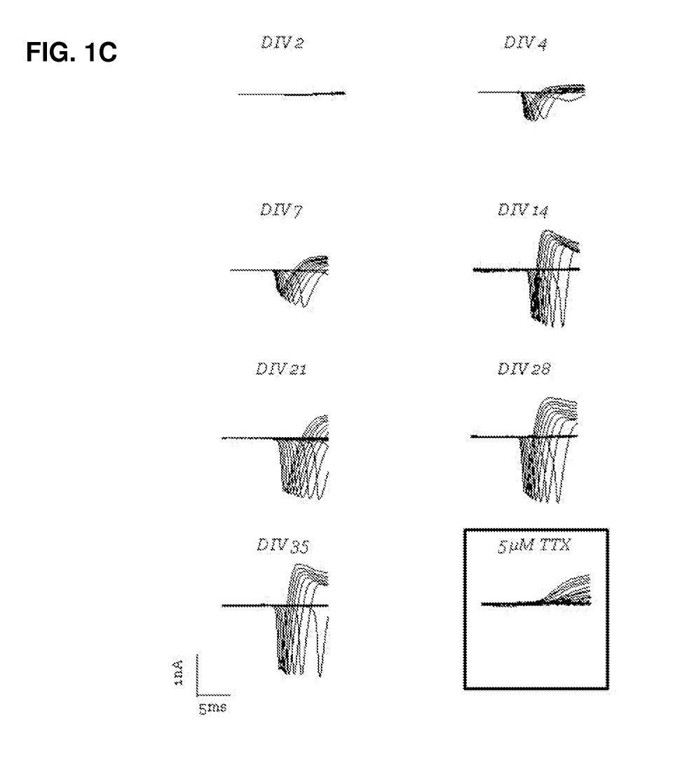

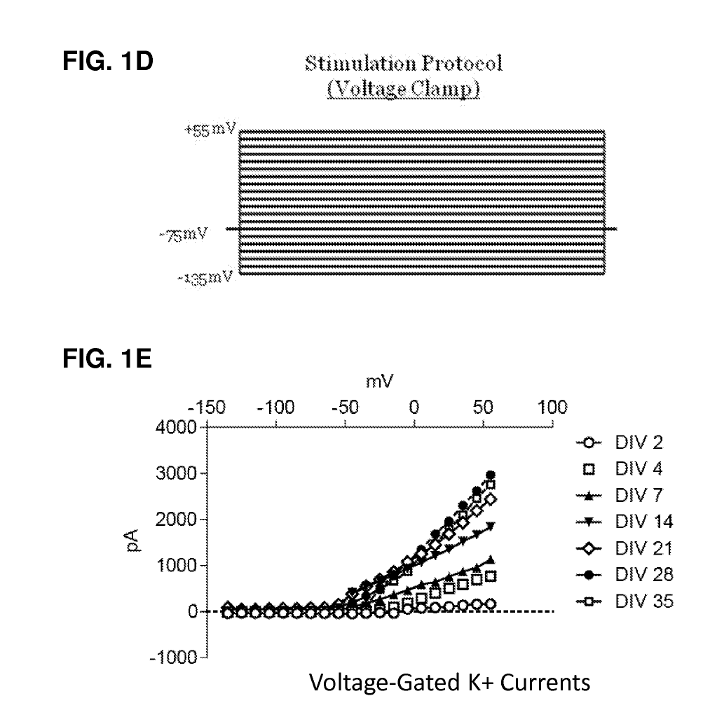

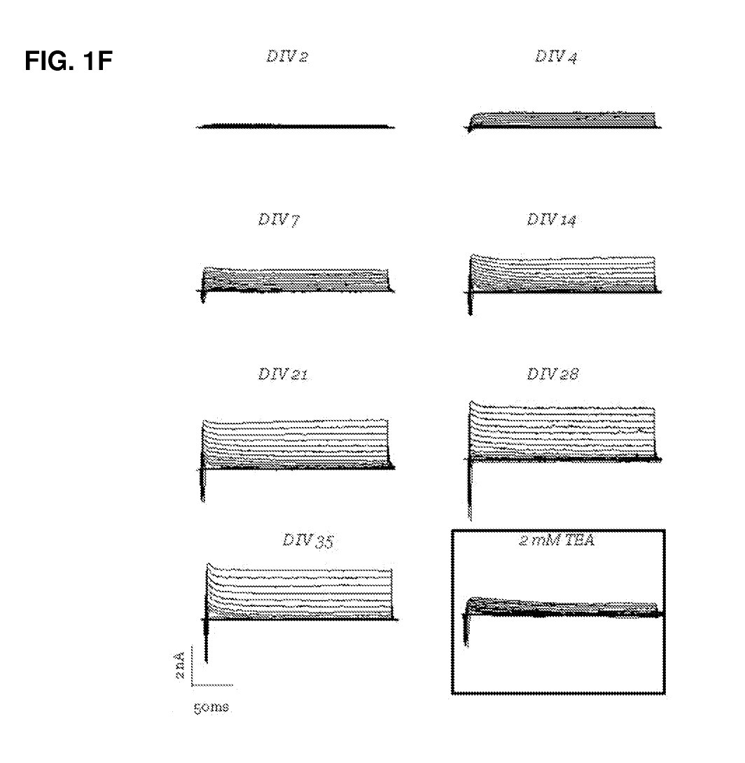

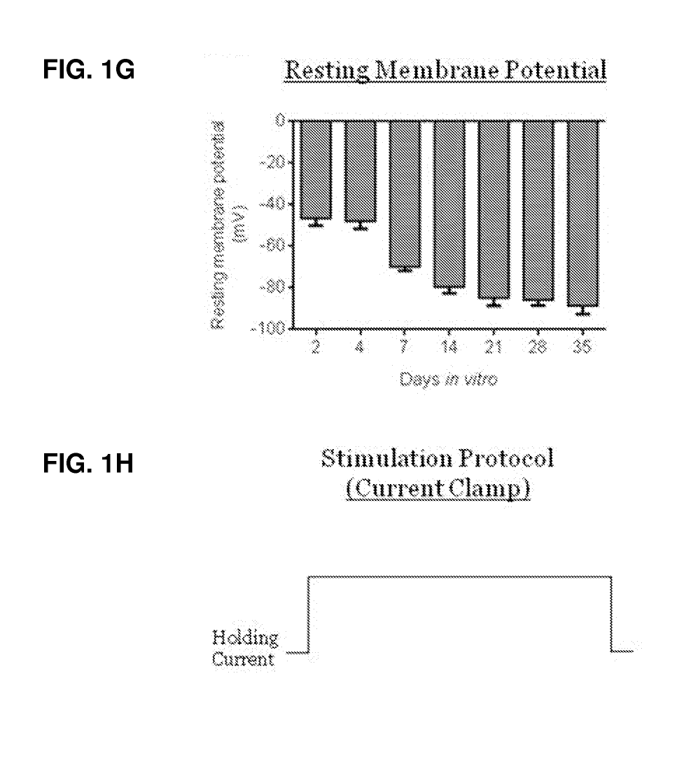

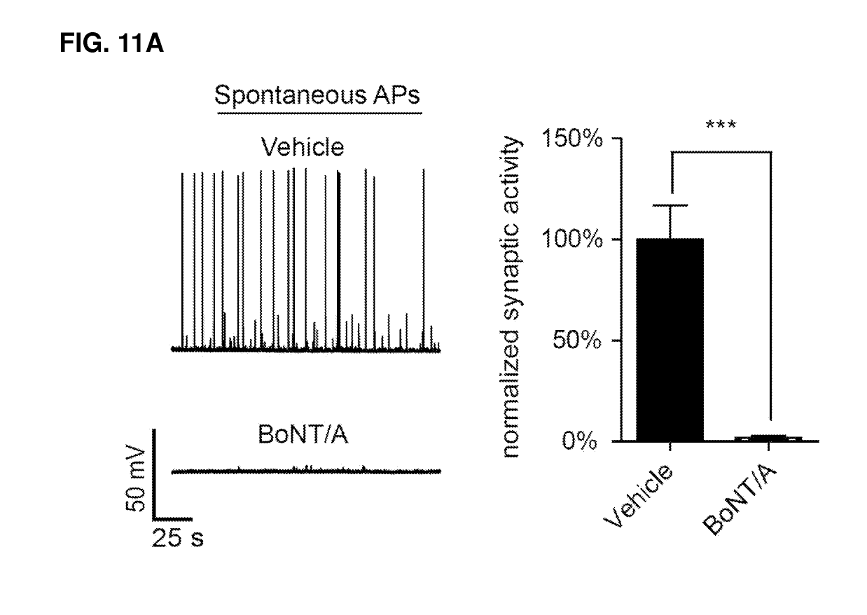

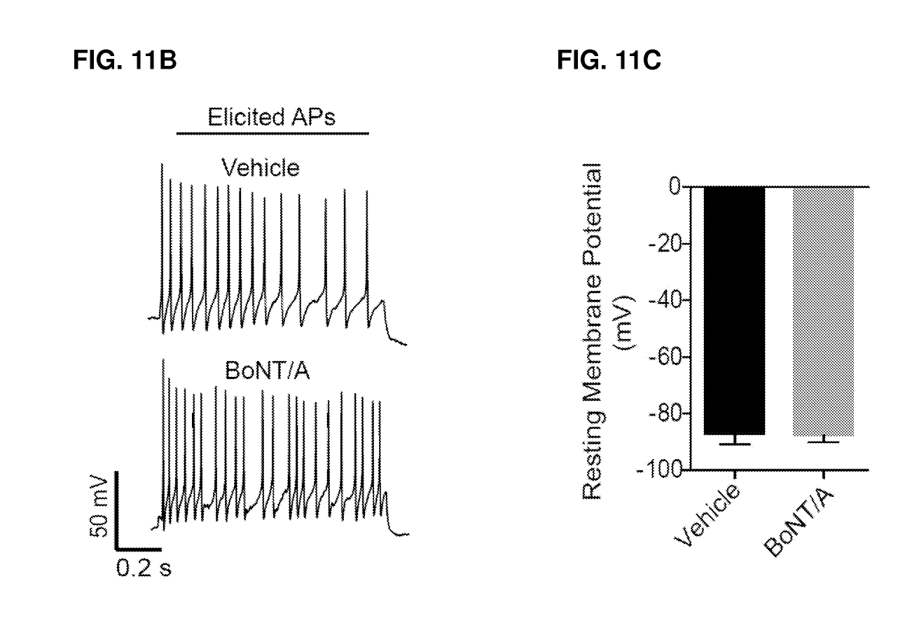

The functional characterization of the maturation-dependent development of intrinsic neuronal properties in ESNs was analyzed. FIG. 1A depicts a schematic stimulation protocol of voltage-clamp recordings with voltage steps of −95 mV to −5 mV (in 5 mV intervals) following a −115 mV pre-pulse for 100 ms. FIG. 1B shows a peak current-voltage plot and representative traces (FIG. 1C) of fast-acting, fast-inactivating sodium channels in DIV2-DIV35 ESNs. FIG. 1D depicts a schematic stimulation protocol of voltage-clamp recordings with incremental voltage steps from −135 mV to +55 mV for 250 ms (in 10 mV intervals). Vm=−75 mV. FIG. 1E shows a peak current-voltage plot and representative traces (FIG. 1F) of a delayed rectifier potassium current in DIV2-DIV35 ESNs. FIG. 1G shows the maturation-dependent development of a negative resting membrane potential in DIV2-DIV 35 ESNs. FIG. 1H shows a schematic stimulation protocol of current-clamp recordings using a depolarizing current injection for 1 s to elicit multiple action potential firings. FIG. 11 shows a representative trace of current-clamp recordings illustrating evoked action potentials in DIV2-DIV35 ESNs.

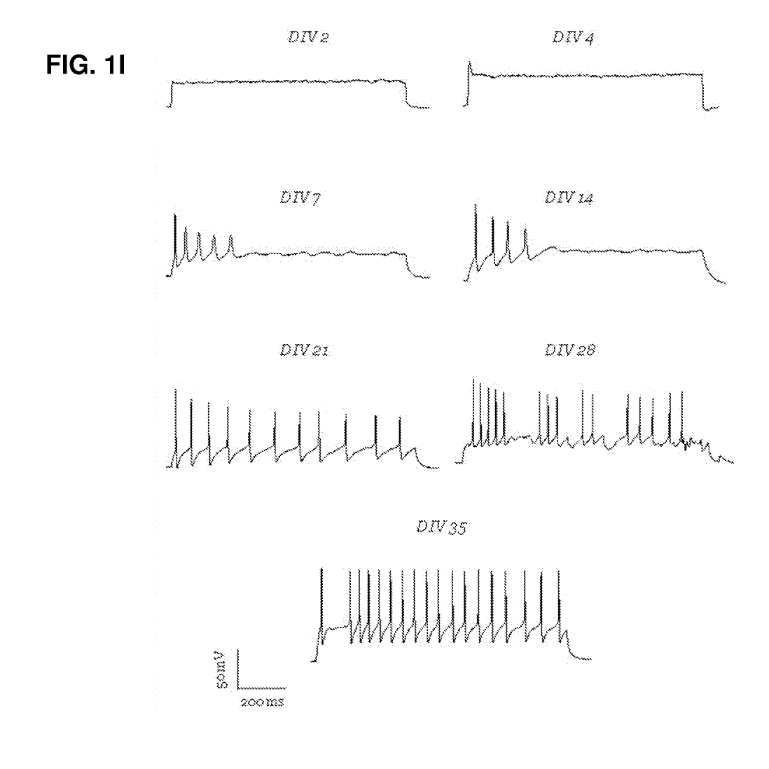

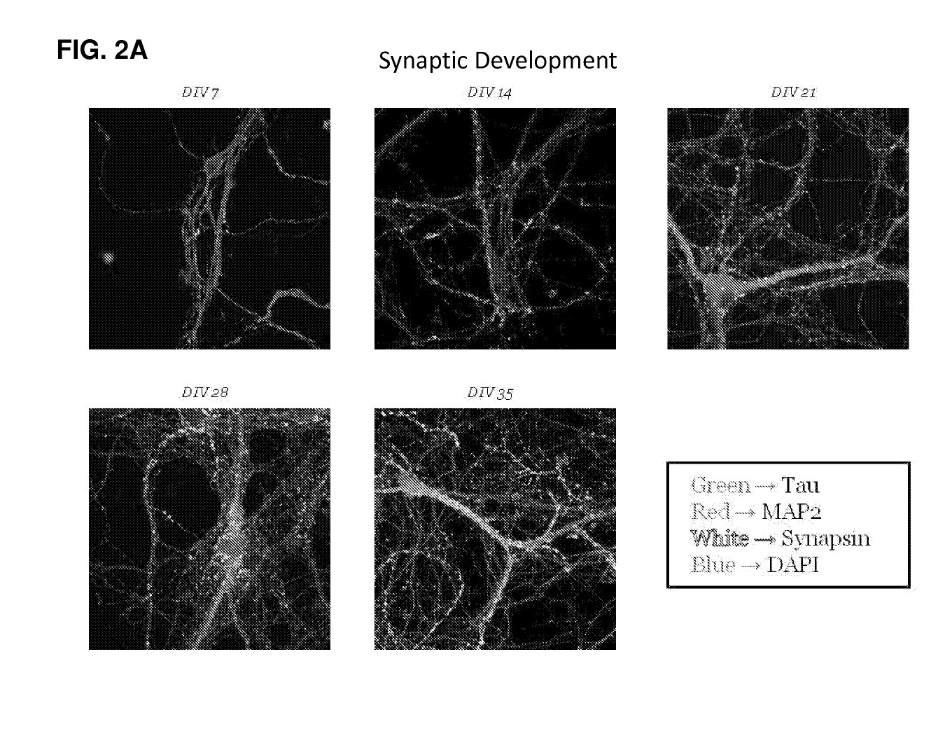

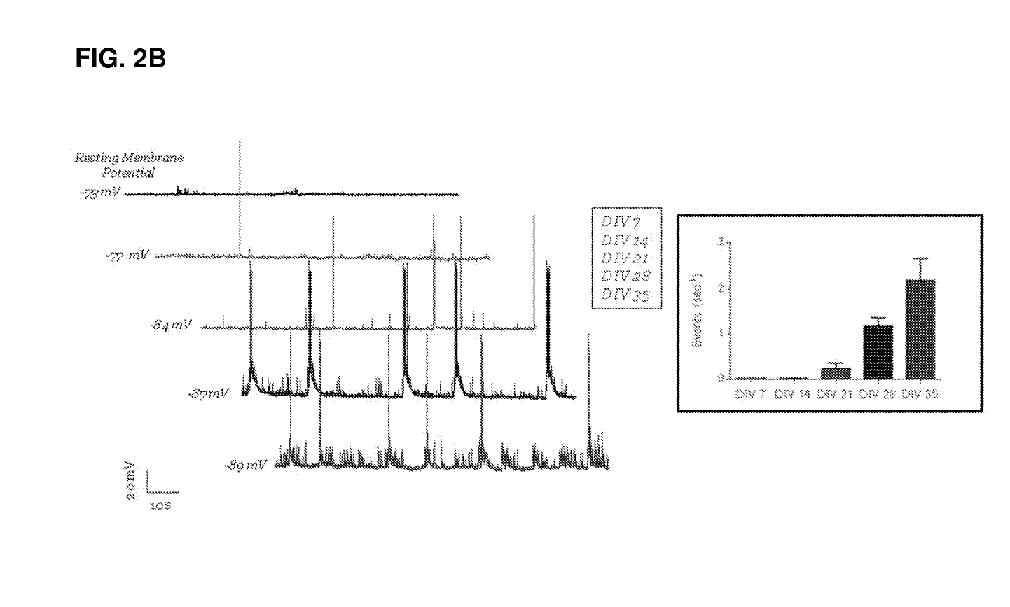

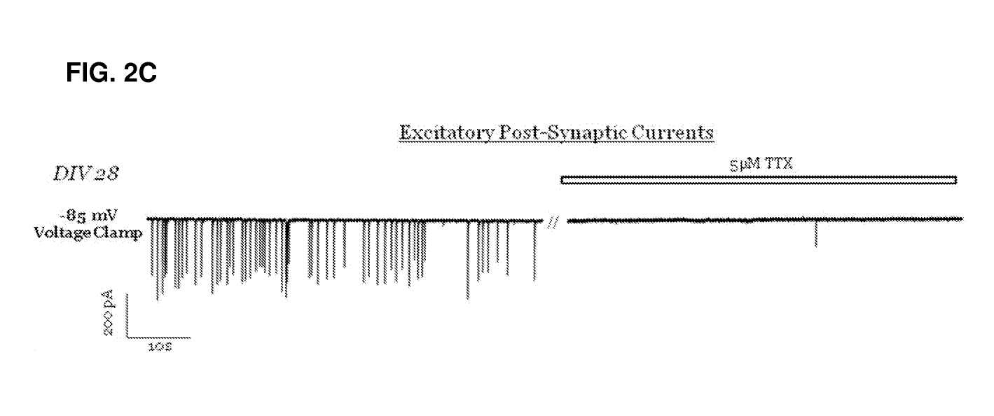

The functionality of the synapses developed by the ESNs was analyzed. Immunocytochemistry performed in ESN cultures from DIV7-DIV35 demonstrated the development of the neuronal arbor and appearance of synaptic puncta at axodendritic interfaces (FIG. 2A). Axons were labeled with Tau (green), dendrites were labeled with MAP2 (red), pre-synaptic compartments were labeled with synapsin (white), and nuclei were stained with DAPI (blue). A representative trace of spontaneous activity and quantification of events per second from continuous current-clamp recordings illustrated the appearance of spontaneous excitatory post-synaptic potentials (EPSPs) and action potentials (APs) from DIV7-DIV35 in ESN cultures (FIG. 2B). Excitatory post-synaptic currents were abundant by DIV28 and were blocked by treatment with the voltage-gated sodium channel antagonist TTX (5 μM) (FIG. 2C).

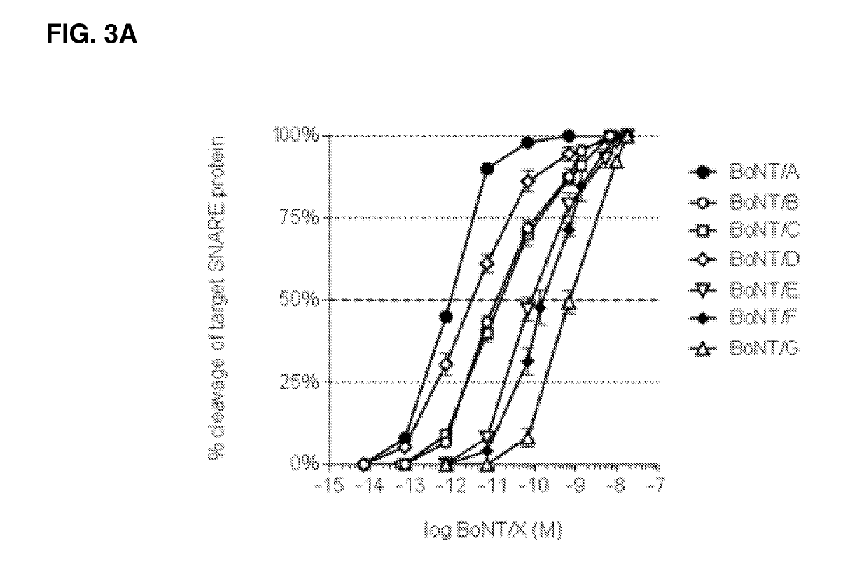

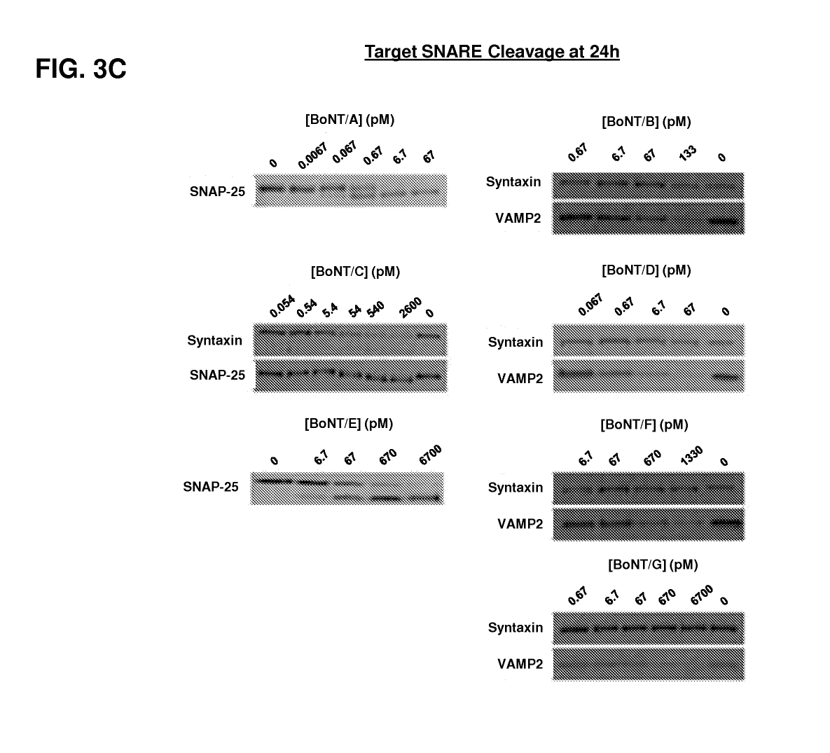

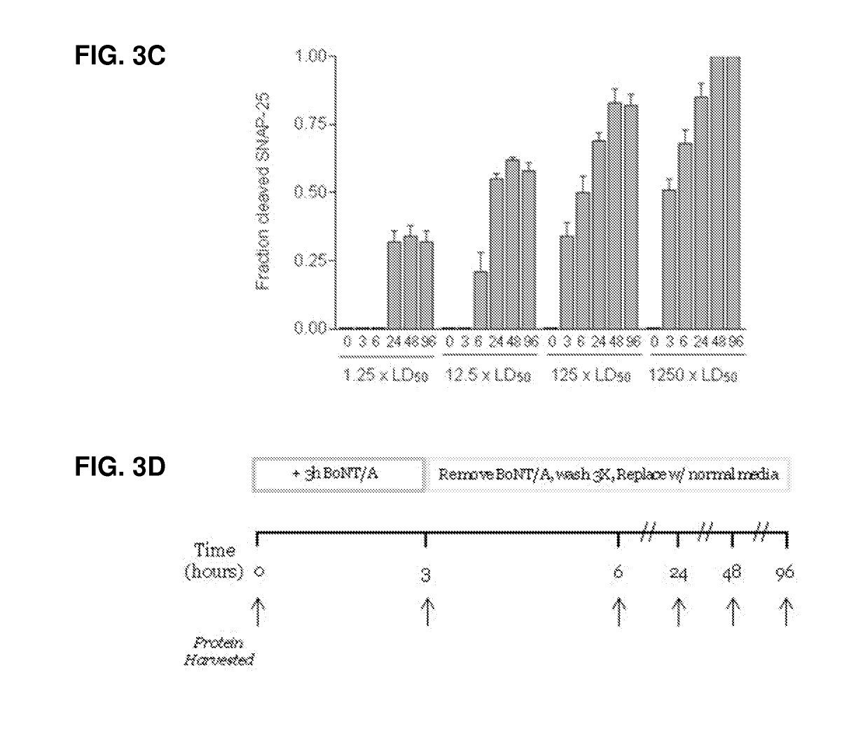

The sensitivity of ESNs to BoNT serotypes was analyzed. ESNs were sensitive to all classical BoNT serotypes, with limits of detection that are less than 1 mouse lethal unit (MLU) for BoNT/A-/E using immunoblot assays (FIG. 3). FIG. 3A shows the quantification of target SNARE cleavage for BoNT serotypes /A-/G. Toxin concentrations were converted to MLUs based on the LD₅₀ values determined by the mouse lethality assay. FIG. 3B shows representative Western blots of target SNARE cleavage at 24 hours following the addition of varying concentrations of BoNT/A-/G. FIG. 3C shows a time- and dose-dependent response of SNAP-25 cleavage following BoNT/A intoxication. FIG. 3D shows a schematic of the experimental design.

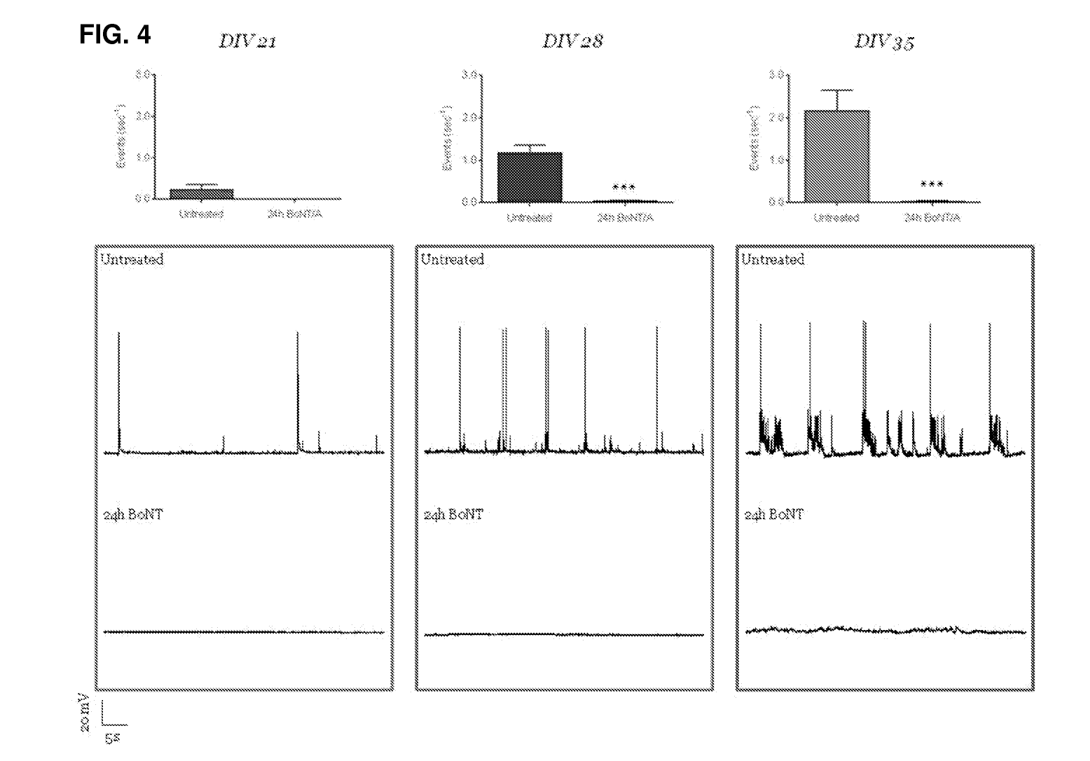

BoNT/A intoxication of ESN synapses can be detected in DIV28+ cultures (FIG. 4). By 24 hours after exposure to 1 mouse lethal unit (5 pg) of BoNT/A, spontaneous activity (events/second) is effectively eliminated in DIV28 and DIV35 ESNs (***=p<0.001 compared to untreated controls). Inhibition is apparent in DIV21 ESNs.

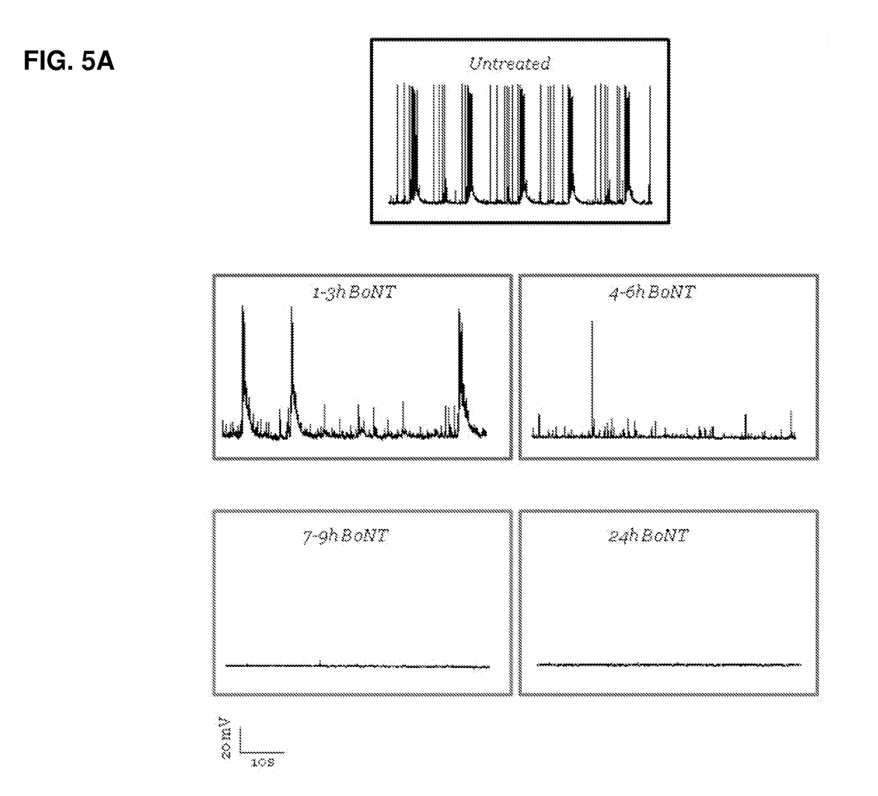

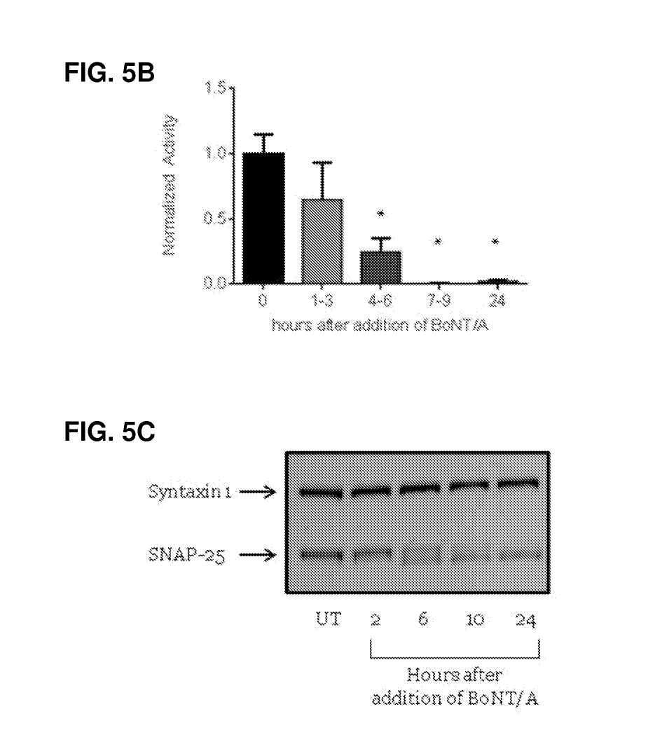

The spontaneous activity of DIV28+ESNs is decreased by as early as 4 hours post-BoNT/A addition (FIG. 5). FIG. 5A shows representative traces of spontaneous activity from current-clamp recordings of DIV28+ following BoNT/A intoxication. FIG. 5B shows quantification of spontaneous activity at indicated duration of BoNT/A exposure. Normalized activity is expressed as a ratio of events/second to untreated controls (*=p of at least <0.05). FIG. 5C shows a Western blot of SNAP-25 cleavage at indicated time points after BoNT/A addition. Note that by 6 h, although spontaneous activity has nearly ceased, only 50% of SNAP-25 is cleaved.

These results indicate that mouse ESNs exhibit maturation-dependent development of intrinsic functional neuronal properties (voltage-gated currents, elicited action potential firing) consistent with primary neuron cultures. Further, ESNs develop a complex network of synaptic connections, which can be functionally detected using whole-cell patch clamp electrophysiology. The ESNs used are highly sensitive to BoNT intoxication. The treatment of these ESNs with BoNT serotypes /A-/G resulted in the specific cleavage of target SNARE proteins in a time- and dose-dependent fashion, with detection at 1 mouse lethal unit possible for all serotypes other than IF and /G. BoNT/A intoxication of DIV28+ESN synapses can be functionally detected using whole-cell patch clamp electrophysiology as early as 4 hours post-treatment. Inhibition of synaptic activity measures the same pathophysiology evoked by BoNT intoxication in vivo and requires all steps of toxin activity (binding, internalization, activation and SNARE cleavage).

### Example 3

### Materials and Methods for Examples 4-6

Reagents.

R1ESCs were obtained from ATCC. Pure botulinum holotoxin serotypes /A (2.5×10⁸ LD50/mg), /B (1.1×10⁸ LD50/mg), were obtained from Metabiologics (Madison, Wis.) at 1 mg/mL in Ca²⁺/Mg²⁺-free phosphate buffered saline, pH 7.4 (PBS), and stored at −30° C. Tetanus Toxin from Clostridium tetani was purchased from List Biological Laboratories (approximate specific activity of 1.5×10⁷ LD50/mg; Campbell, Calif.). 5-fluoro-2′-deoxyuridine (FUDR), uracil-1-β-D-ribofuranoside (uridine) and arabinocytidine (ara-C) were purchased from Sigma-Aldrich (St. Louis, Mo.)

ESC Culture and Neuronal Differentiation.

ESNs were differentiated from suspension-cultured mouse ESCs as previously described (Hubbard et al. 2012) with the following changes: ESNs were treated with mitotic inhibitors (30 μM FUDR, 70 μM uridine, 5 μM ara-C) diluted in Neurobasal medium (NBA) with 1× B27 supplements (Invitrogen, Carlsbad, Calif.) from DIV 8-12 in a standard incubator at 7.5% CO₂, after which they were washed and transferred to a Coy chamber (Coy Laboratory Products, Grass Lake, Mich.) also at 7.5% CO₂. From DIV 13 and on, half-media changes were conducted on a weekly basis using CO₂-equilibrated media. For intoxication, BoNTs and TeNT were diluted to the appropriate concentrations in CO₂-equilibrated B27/NBA, added to neurons for 24 h and ESNs were washed and removed from the Coy chamber for analysis.

Immunoassays.

For detection of SNARE protein cleavage, ESN cultures were washed twice with PBS, lysed with 250 μL denaturing cell extraction buffer (Life Technologies) and clarified by centrifugation through a Qiashredder (Qiagen, Valencia, Calif.). Total protein concentration was determined by bicinchoninic acid (BCA) analysis (Thermo Scientific, Rockford, Ill.) and 15 μg of total protein was separated on a 12% Nupage gel (Life Technologies) with MOPS running buffer. Gels were transferred to PVDF and probed for SNARE proteins with a mouse anti-SNAP-25 antibody (SMI81; Covance, Gaithersburg, Md.), a mouse anti-VAMP2 antibody (Synaptic Systems, Gottingen, Germany), and a mouse anti-Syntaxin-1 (Synaptic Systems) diluted 1:1000 in TBS Superblock with 0.05% Tween-20 (TBST; Life Technologies). Proteins were visualized with goat anti-mouse Alexa-488 labeled antibodies diluted 1:2500 in TBST and imaged with a Versadoc MP4000 (Bio-Rad, Hercules, Calif.). Since cleaved VAMP2 is not detectable by immunoblot, we normalized the VAMP2 signal in each lane to the corresponding signals for syntaxin-1a (stx1a) and SNAP25 (Hubbard, et al 2012). Isolation of synaptosomal and cytosolic fractions was achieved using Syn-Per (Pierce)) For ICC, DIV 25-32 ESNs plated on coverslips were fixed with 4% paraformaldehyde for 15 min at room temperature and blocked and permeabilized for 10 min in PBS with 0.1% saponin and 3% bovine serum albumin (BSA) (PBSS). Coverslips were incubated for 1 h with primary antibodies against VAMP2, syntaxin-1 and SNAP25 (mouse); diluted 1:1000 in PBSS, washed three times with PBSS and incubated for 1 h with Alexa-labeled secondary antibodies (Invitrogen) diluted 1:500 in PBSS. Coverslips were washed three times in PBSS and mounted with Prolong Gold DAPI mounting media (Life Technologies). Images were collected with a Zeiss LSM 700 confocal microscope.

Electrophysiology.

Whole-cell patch-clamp electrophysiology was performed. Briefly, for mEPSC detection and current-clamp recordings, recording pipettes were filled with an intracellular recording buffer containing (in mM): 140 K-gluconate, 5 NaCl, 2 Mg-ATP, 0.5 Li-GTP, 0.1 CaCl₂, 1 MgCl₂, 1 ethylene glycol-bis (b-aminoethyl ether)-N,N,N,N-tetraacetic acid (EGTA) and 10 HEPES. Cultures were bathed in an extracellular recording buffer (ERB) containing (in mM): 140 NaCl, 3.5 KCl, 1.25 NaH₂PO₄, 2 CaCl₂, 1 MgCl₂, 10 Glucose, 10 HEPES. All buffers were adjusted to pH of 7.3 with NaOH or KOH and an osmolarity of 315±10 mOsm with glucose. For mEPSC detection, ERB was supplemented with 5 μM tetrodotoxin and mEPSCs were quantified using MiniAnalysis (Synaptosoft, Inc., Decatur, Ga.) with recommended detection parameters.

Statistical Analysis.

Graphpad Prism v6.01 (Graphpad Software, La Jolla, Calif.) was used to calculate EC50 values from densitometry of western blot images or from mEPSC rates quantified via MISA using a four-parameter sigmoidal model. Differences among means were determined and calculated with the Student's t-test (for binary comparisons) or one-way ANOVA followed by a Dunnett post-hoc test to the appropriate reference sample. Data are presented as mean±SEM unless otherwise noted. * indicates a P<0.05. ** indicates a P<0.01. *** indicates a P<0.001.

### Example 4

### Networked Populations of ESNs are Sensitive to BoNT/A, /B and TeNT

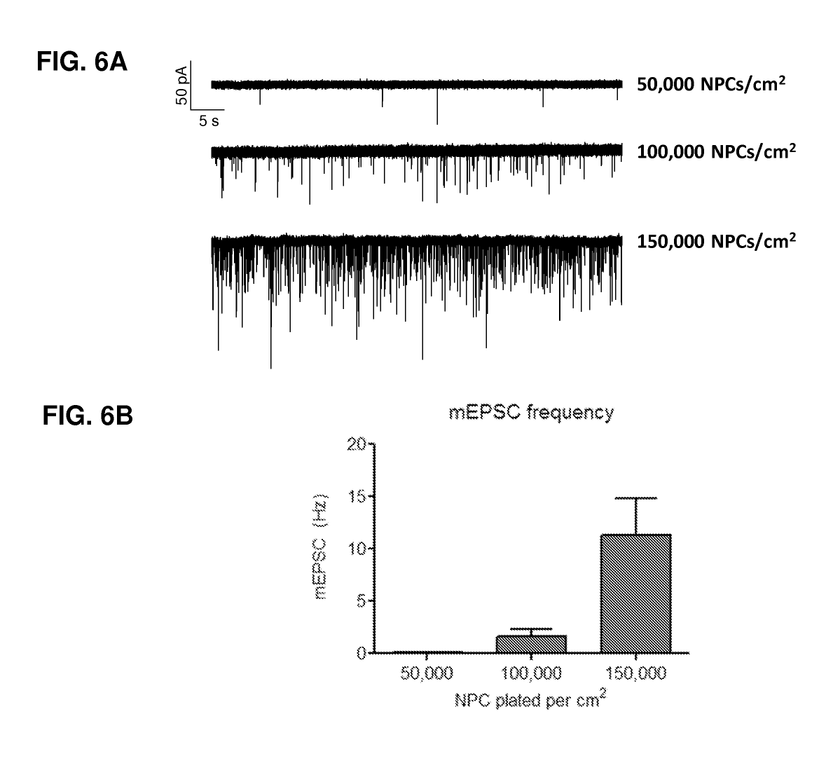

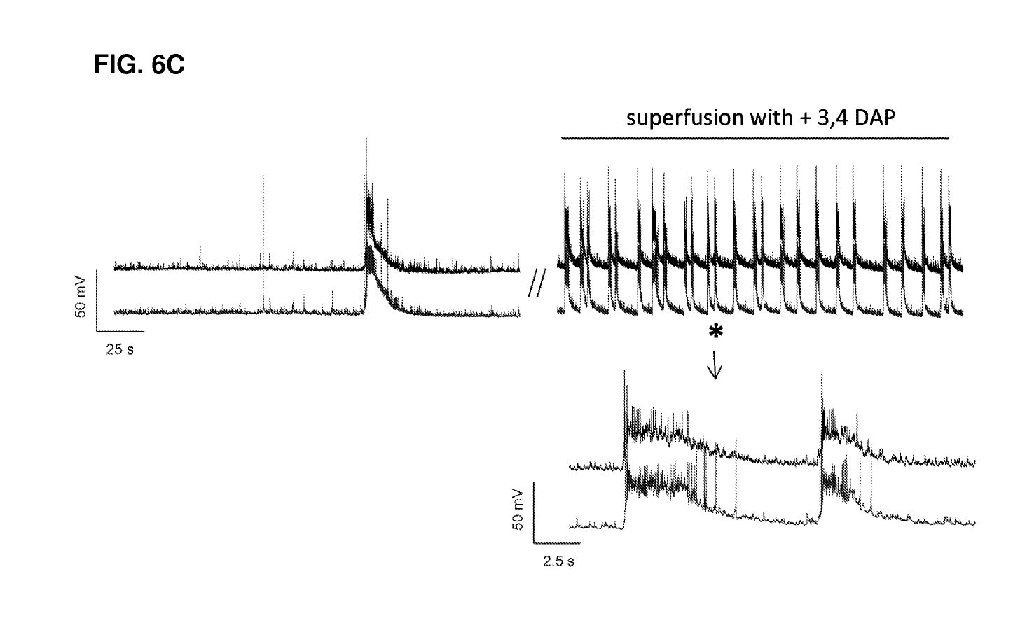

Measurements of synaptic inhibition require neuronal populations that form functional synapses with basal rates of activity. To determine a plating density that was conducive to measuring synaptic activity, neurons were plated at 50,000, 100,000 or 150,000 neurons/cm² and mEPSC frequencies were measured at DIV 21 (FIG. 6A). Neurons plated at 150,000 cells/cm² produced the highest frequency of synaptic activity, and exhibited extensive formation of synaptic puncta at regions of axodendritic interface (FIG. 6B). To confirm emergence of network behaviors, EPSPs were evaluated in simultaneous dual intracellular recordings prior to and following addition of 3,4-diaminopyridine (3,4-DAP), which blocks K+ efflux through voltage-gated potassium channels and increases AP-induced neurotransmitter release. While neurons initially exhibited spontaneous network behaviors, perfusion with 3,4-DAP elicited a rapid transition to a synchronized bursting behavior, confirming synaptic coupling and network emergence (FIG. 6C).

FIG. 6 shows the effect of plating density on activity at DIV 23+. ESNs were harvested 1 day after differentiation and re-plated at the listed concentrations and spontaneous miniature excitatory post-synaptic currents were measured in the presence of tetrodotoxin and GABAR-A and GABAR-B antagonists at DIV 24. Representative traces of spontaneous mEPSC activity (FIG. 6A). Quantitation of spontaneous mEPSCs (n=8 per plating density) (FIG. 6B). Mean+SEM is plotted; **=p<0.05; ***=p<0.001. Simultaneous patch clamp demonstrates network behavior (FIG. 6C). Two neurons (black, top and blue, bottom traces) were simultaneously patched and evaluated for spontaneous network activity. Neurons showed spontaneous distinct as well as coordinated activity. Superfusion of the excitatory 3,4-diaminopyridine elicited a rapid transition to a synchronized bursting behavior, confirming synaptic coupling and network emergence.

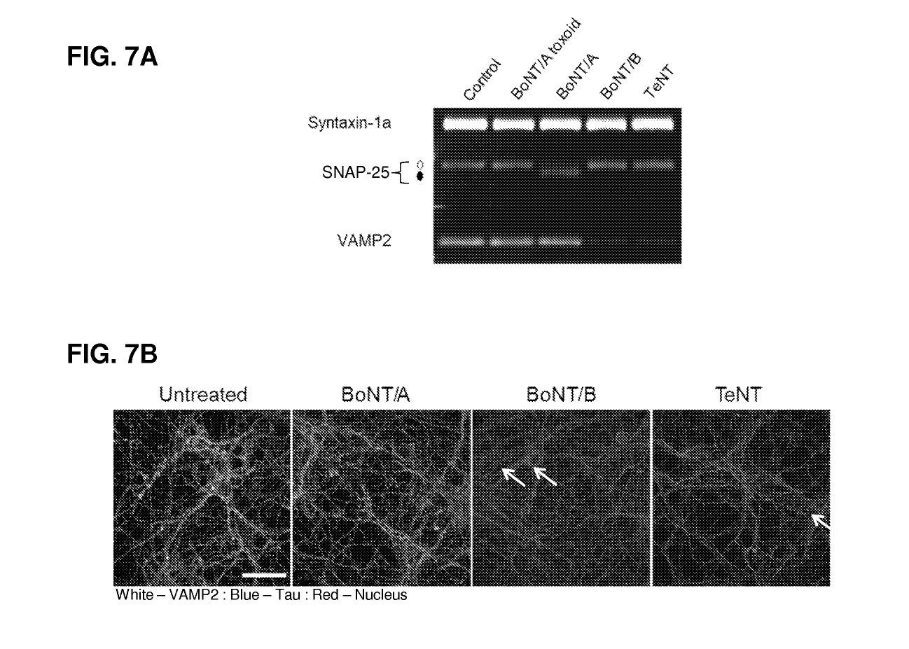

To confirm that networked ESNs were sensitive to intoxication, ESNs were exposed to BoNT/A (67 pM), non-toxic BoNT/A toxoid (670 pM), BoNT/B (175 pM) or TeNT (175 pM) for 24 h, and SNARE protein cleavage was evaluated by immunoblot and immunocytochemistry. Treatment with BoNT/A cleaves SNAP25, converting the 25 kDa intact protein to a 24 kDa cleaved SNAP25 (cSNAP25) fragment, while the formalin-inactivated BoNT/A toxoid had no effect on SNARE protein integrity (FIG. 7A). In contrast, cleaved VAMP2 is rapidly cleared from the neuron, so immunoassays to detect the activity of BoNT/B and TeNT are based on the loss of VAMP2 signal. Treatment with 175 pM BoNT/B or 175 pM TeNT resulted in the near complete loss of VAMP2 signal by western blot. Consistent with the immunoblot data, immunocytochemistry (ICC) demonstrated that SNAP-25, VAMP2 and syntaxin-1a have distinct distributions, with VAMP2 and syntaxin-1a co-localized in puncta whereas SNAP25 has an axolemmal distribution (FIG. 7B). While treatment with BoNT/A did not disrupt SNAP25 reactivity, treatment with BoNT/B or TeNT caused the near complete loss of VAMP2 signal, with remaining VAMP2 primarily associated with the soma. These data confirmed that networked ESNs are sensitive to BoNT/A and /B, and demonstrated for the first time that ESNs are susceptible to TeNT.

FIG. 7 shows that DIV 23+ESNs are sensitive to clostridial neurotoxins. FIG. 7A shows a representative western blot showing SNARE protein cleavage following intoxication with different clostridial neurotoxins. Note that addition of 175 pM BoNT/B or TeNT results in the loss of VAMP2 signal, whereas intoxication with 67 pM BoNT/A results in a cleaved SNAP25 band. Neither vehicle-controls nor 670 pM formalin-inactivated toxoid results in changes in SNAP-25 or VAMP2. The intact SNAP-25 band is denoted by hollow circle; BoNT/A-cleaved SNAP-25 band is denoted by filled circle. FIG. 7B shows immunocytochemistry of ESNs exposed to 67 pM BoNT/A (which cleaves SNAP25), 175 pM BoNT/B or 175 pM TeNT for 24 h. In comparison to controls (untreated and 2000 U/mL BoNT/A), which showed no apparent difference in VAMP2 (white) expression, intoxication with 175 pM (2000 U/mL) BoNT/B or 175 pM TeNT caused the extensive loss of VAMP2 immunoreactivity. In most cases, the remaining VAMP2 (white arrows) is located in puncta close to the soma.

### Example 5

### Functional Measurements of Synaptic Inhibition by BoNT/A are More Sensitive than the MLA or Immunoassays

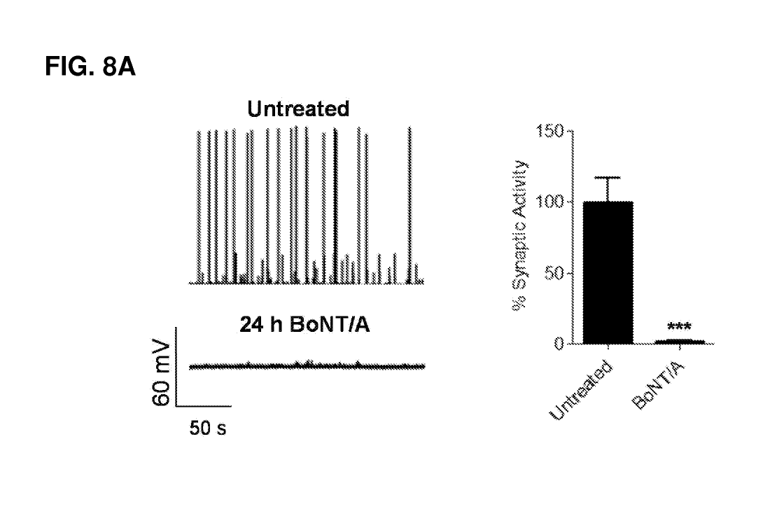

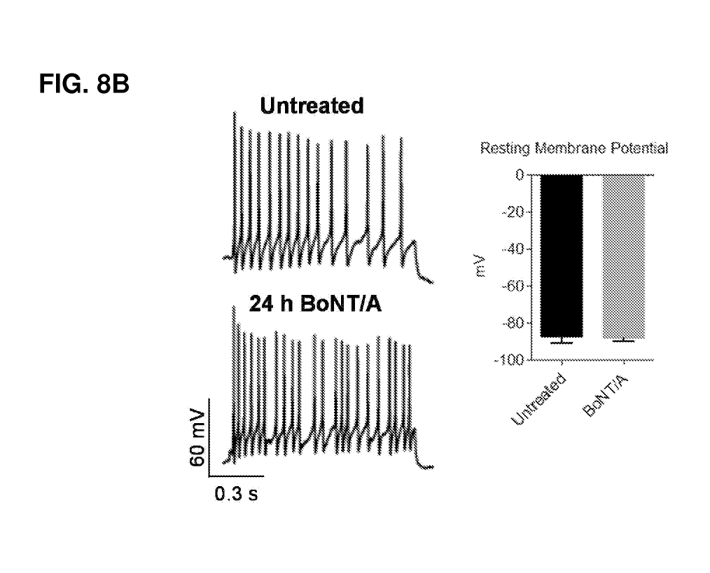

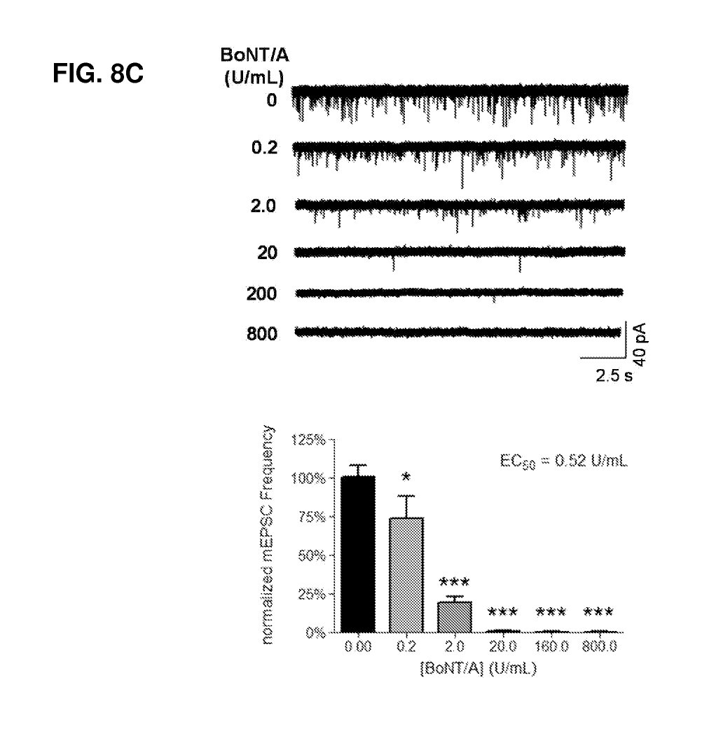

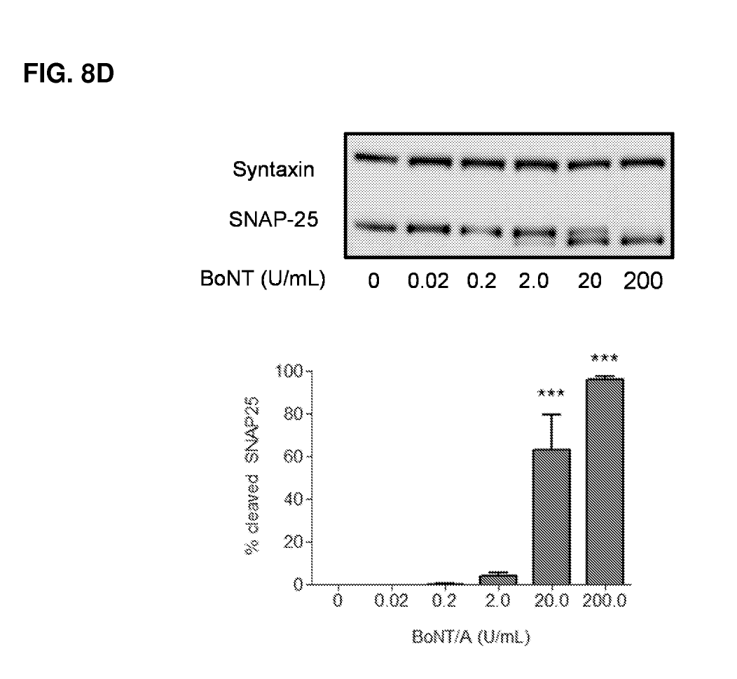

We developed the measured inhibition of synaptic activity (MISA) assay to quantify the effect of intoxication on neurotransmitter release with single-synapse resolution. MISA is based on whole-cell patch-clamp electrophysiological quantitation of mEPSC frequency in neurons exposed to CNTs and normalized against age-matched vehicle controls. To compare the sensitivity of MISA to the MLA and SNARE cleavage assay, we evaluated synaptic inhibition by MISA 20 h after intoxication with 0.02-200 U/mL BoNT/A (FIG. 8A). Treatment with 0.2 U/mL BoNT/A (equivalent to 5.4 fM) caused a significant reduction of mEPSCs to 26.4±14.6% of controls, with an EC50 value of 0.52 U/mL (R2=0.67). To confirm that the loss of spontaneous synaptic activity was not due to alteration of intrinsic excitability, neurons exposed to 200 U/mL BoNT/A for 24 h were still able to produce voltage-gated responses (FIG. 8B), suggesting that the loss of mEPSCs was specifically attributable to BoNT/A-mediated synaptic inhibition. Cell viability assays further confirmed the absence of neurotoxicity in treated cultures (FIG. 8C). The EC50 value of SNAP25 cleavage by immunoassay was calculated to be 14.4 U/mL (95% Cl; R2=0.97), or approximately 30-fold less sensitive than MISA (FIG. 8D; Table 1). Notably, cleavage of just 4.7% of total cellular SNAP25 was sufficient to reduce synaptic activity by 74.7% in the 2 U/mL treatment, whereas no apparent cleavage of SNAP25 was apparent at 0.2 U/mL, even though mEPSC frequency was inhibited by 26.4%. These data confirm that detection of BoNT/A in ESNs using MISA is significantly more sensitive and faster than either the MLA or cleavage immunoassays. Furthermore, they indicate that the cleavage of a small sub-population of total cellular SNAP25 is sufficient to prevent neurotransmitter release.

FIG. 8 shows that a 20 h treatment of ESNs with botulinum neurotoxin A results in loss of synaptic activity. FIG. 8A shows representative current-clamp recording of spontaneous action potentials (APs) and excitatory post-synaptic potentials (EPSPs) in untreated vs. 24 hour BoNT/A (800 U/mL) treated DIV 23+ESN cultures (left panel). Quantification of spontaneous events (right panel) in untreated vs. 24 hour BoNT/A (800 U/mL) treated ESNs (n>18 for each treatment). FIG. 8B shows treatment of ESNs with BoNT/A (800 U/mL) for 24 hours does not alter intrinsic neuronal properties, including the ability to fire repeated APs in response to depolarizing current injection (left panel) or the ability to maintain a negative resting membrane potential (right panel; n>18 for each treatment). FIG. 8C shows the dose response of BoNT/A (0.2-800 U/mL) after 24 hours of treatment as assessed by mEPSC frequency using the measured inhibition of synaptic activity assay (MISA). Top panel=representative voltage-clamp trace segments, bottom panel=quantification of mEPSC frequency (n=20 for controls; n>10 for each dose). FIG. 8D shows treatment of ESNs results in cleavage of SNAP-25 as visualized by gel mobility shifts (top panel=representative Western blot, bottom panel=quantification; n=4). Note decreased sensitivity of Western blot vs. MISA.

### Example 6

### TeNT and BoNT/B Addition Results in the Dose-Dependent Loss of Synaptic Activity at 24 h

All CNTs proteolyze SNAP25 or VAMP2. To determine whether the MISA approach was also compatible with CNTs that cleave VAMP2, we measured synaptic inhibition following intoxication by BoNT/B and TeNT.

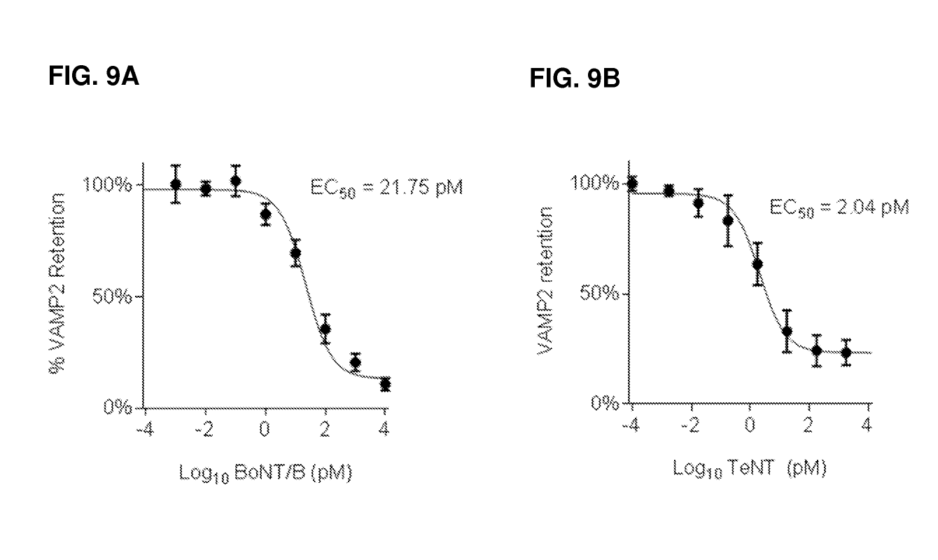

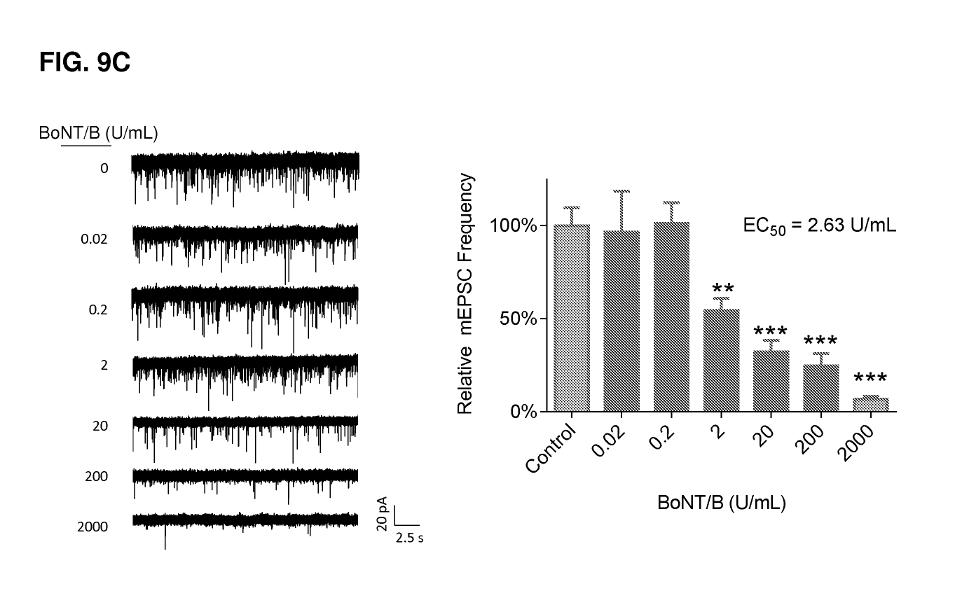

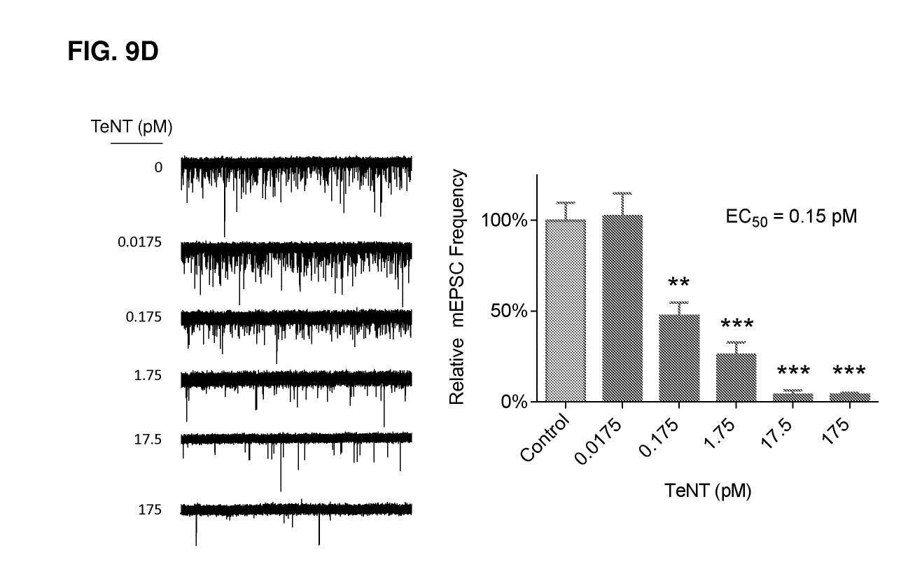

Treatment of DIV 25 ESNs for 24 h with TeNT concentrations ranging from 1.75 fM to 17.5 nM and BoNT/B concentrations ranging from 0.01-200 U/mL resulted in a dose-dependent loss of VAMP2, with TeNT EC50 values of 2.04 pM (95% CI: 0.97 to 4.29, R2=0.91; FIG. 9A) and BoNT/B EC50 values of 21.75 pM (95% CI: to, R2=; FIG. 9B). Notably, exposure to CNTs at concentrations that were three orders of magnitude above the EC50 value did not result in a complete loss of VAMP2 signal, suggesting that a reserve of VAMP2 remained inaccessible to activated light chains.

To measure the comparable sensitivity of MISA against the immunoblot assays, we used MISA to measure the functional consequence of intoxication 20 h after addition of 0.2-200 U/mL (12.2-12,222 fM) BoNT/B or 17.5-17,500 fM TeNT (FIG. 9C, 9D). In both cases, intoxication resulted in a dose-dependent inhibition in mEPSC frequency. Consistent with the immunoblot data suggesting a reserve of VAMP2 even after intoxication with high doses of CNTs, a low-level of mEPSC activity remained even at the highest BoNT/B or TeNT doses. Nonetheless, the use of MISA to measure BoNT/B or TeNT was significantly more sensitive than immunoblot assays, as BoNT/B intoxication reduced mEPSC activity with an EC50 value of 0.14 pM and TeNT intoxication reduced mEPSC activity with an EC50 value of 0.15 pM. The immunoblot and MISA data are summarized for all CNTs on Table 1.

TABLE 1
Comparison of MISA and immunoblot EC₅₀ values

| | |
|---|---|
| | Immunoblot |
| MISA EC₅₀ | EC₅₀ |

| Toxin | U/mL | pM | U/mL | pM |
|---|---|---|---|---|
| BoNT/A | 0.52 | 0.014 | 14.40 | 0.39 |
| BoNT/B | 2.63 | 0.14 | 357.09 | 21.75 |
| TeNT | 0.33 | 0.15 | 4.57 | 2.04 |

In an attempt to determine whether the uncleaved VAMP2 fraction was localized within the pre-synaptic compartment, we evaluated the distribution of intact VAMP2 protein in synaptosomal versus whole cell lysates 24 h after exposure to 1.75 nM TeNT, 2000 U/mL BoNT/B or 200 U/mL BoNT/A. VAMP2 was enriched approximately 4-fold in the synaptosomal fraction of control ESNs and neurons exposed to BoNT/A, whereas intact VAMP2 was only observed in the cytosolic fraction in neurons exposed to BoNT/B or TeNT. This confirmed that the CNT-resistant VAMP2 population was extra-synaptic and indicated that synaptic VAMP2 was efficiently cleaved by BoNT/B and TeNT.

FIG. 9 shows that intoxication of DIV 23+ESNs with BoNT/B or TeNT causes loss of VAMP2 and synaptic inhibition by 20 h. FIGS. 9A and 9B show dose-response data for VAMP2 loss after 20 h intoxication with BoNT/B (FIG. 9A) or TeNT (FIG. 9B). FIGS. 9C and 9D show representative traces (top) and average mEPSC frequencies measured (bottom) by MISA at 20 h after addition of BoNT (FIG. 9C) or TeNT (FIG. 9D). *=p<0.05; **=p<0.01; ***=p<0.001.

### Example 7

### Materials and Methods for Examples 8-12

ESN Culture and BoNT/A Intoxication.

R1 murine embryonic stem cells were maintained and differentiated into ESNs. Experiments were conducted on ESNs at DIV 24-34 (DIV 24+). Throughout the study, data were collected from 10 independent ESN differentiations over 6 months, with no apparent change in functional response to intoxication. Independent ESN cultures were checked for synaptic functionality using whole-cell patch clamp recordings to ensure complex mEPSC frequency exceeded 4.0 Hz prior to experimentation. 10 out of 11 differentiations passed this criteria and were included in this study. BoNT/A1 (specific activity=2.5×10⁸ U; Metabiologics, Madison, Wis.) was diluted in extracellular recording buffer and added directly to DIV 24⁺ ESNs at a concentration of 20 pM for the indicated amount of time.

Electrophysiology.

Whole-cell patch clamp recordings were performed. Briefly, for mEPSC detection and current-clamp recordings, recording pipettes were filled with an intracellular recording buffer containing (in mM): 140 K-gluconate, 5 NaCl, 2 Mg-ATP, 0.5 Li-GTP, 0.1 CaCl₂, 1 MgCl₂, 1 ethylene glycol-bis (b-aminoethyl ether)-N,N,N,N-tetraacetic acid (EGTA) and 10 HEPES. For mIPSC detection, recording pipettes were filled with an intracellular recording buffer containing (in mM): 140 CsCl, 5 NaCl, 2 Mg-ATP, 0.5 Li-GTP, 0.1 CaCl₂, 1 MgCl₂, 1 EGTA, and 10 HEPES. Cultures were bathed in an extracellular recording buffer (ERB) containing (in mM): 140 NaCl, 3.5 KCl, 1.25 NaH₂PO₄, 2 CaCl₂, 1 MgCl₂, 10 Glucose, 10 HEPES. All buffers were adjusted to pH of 7.3 with NaOH or KOH and an osmolarity of 315±10 mOsm with glucose prior to recording. For mEPSC and mIPSC recordings, ERB was supplemented with 5 μM tetrodotoxin. For isolated mEPSCs, 10 μM bicuculline and 1 μM CGP 55845 were added to ERB. For isolated mIPSCs, 50 μM APV and 10 μM CNQX were added to ERB. For current-clamp recordings, spontaneous EPSPs and APs were detected using MiniAnalysis (Synaptosoft, Inc., Decatur, Ga.) with the following settings: threshold, 5 mV; period to search for local max, 10,000 μs; time before peak, 10,000 μs; period for decay, 4000 μs; fraction of peak to decay, 0.37; period to average baseline, 1000 μs; area threshold, 50; number of points to average peak, 1; direction, positive. For AP bursting quantification, software recommended AP detection parameters were used with the following settings: threshold, 25 mV; period to search for local max, 10,000 μs; time before peak, 10,000 μs; period for decay, 4000 μs; fraction of peak to decay, 0.37; period to average baseline, 1000 μs; area threshold, 50; number of points to average peak, 1; direction, positive. Secondary burst analysis was performed with the following settings: minimum number of consecutive events to be considered a burst, 2; maximum interevent interval between two events, 1 s. A 10 minute baseline recording was captured, with the 5 minutes immediately preceeding superfusion serving as control values. For mEPSC and mIPSC detection, ESNs were voltage-clamped to −70 mV and recorded continuously for at least 4 minutes. mEPSCs and mIPSCs events were detected with MiniAnalysis using software recommended mEPSC and mIPSC detection parameters. To account for the increased background noise during mIPSC recordings, an amplitude of 7.5 times the background signal was used as a threshold for event detection of each recording. For agonist-induced iGluR and GABAR currents, neurons were perfused using a three barrel Fast Step system (Warner Instruments, Hamden, Conn.). For long-term current clamp recordings, vehicle or BoNT/A was gradually perfused into ERB using gravity-fed plastic tubing over the course of 3 to 4 minutes until final concentration was reached.

Immunoassays.

Protein from ESN cultures for Western blot detection was harvested and separated by SDS-PAGE. Antibodies used for Western blotting in this study include SNAP-25 (Covance, Princeton, N.J.) and syntaxin (Synaptic Systems, Gottingen, Germany). For immunocytochemistry, ESNs on 18-mm coverslips were fixed in 4% paraformaldehyde, stained, and visualized by microscopy. Antibodies used in this study include anti-Tau (Synaptic Systems), anti-MAP2 (Synaptic Systems), anti-SNAP-25 (Covance), anti-cleavage-specific SNAP-25 (Research and Diagnostic Antibodies, Las Vegas, Nev.), anti-synapsin (Synaptic Systems), anti-vGluT2 (Synaptic Systems), anti-gad1/gad67 (Synaptic Systems).

Statistical Analysis.

Statistical significance among means was determined using one-way ANOVA testing, and P values were calculated against controls with the Dunnett's post-hoc test. Statistical significance between grouped means was determined using two-way ANOVA testing, and P values were calculated between means with Bonferroni's post-hoc test. For binary comparisons of means, the Student's t-test was used. For quantification of glutamatergic vs. GABAergic synapses, a Shapiro-Wilk normality test was first performed to confirm that data were normally distributed followed by a one-sample t-test comparing each population against a theoretical mean of 50%. Unless otherwise stated, all quantitative data are presented as mean±the standard error of the mean, with the following markers of statistical significance: * indicates a P<0.05; ** indicates a P<0.01; *** indicates a P<0.001.

### Example 8

### ESN Cultures Produce Excitatory and Inhibitory Synapses with Emergent Network Responses

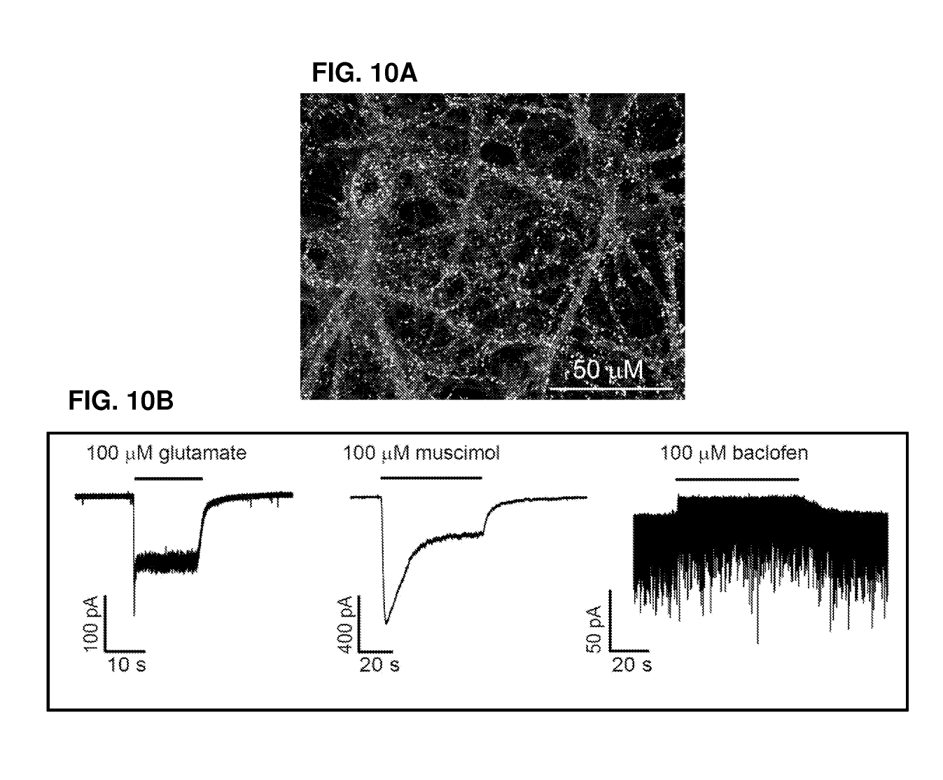

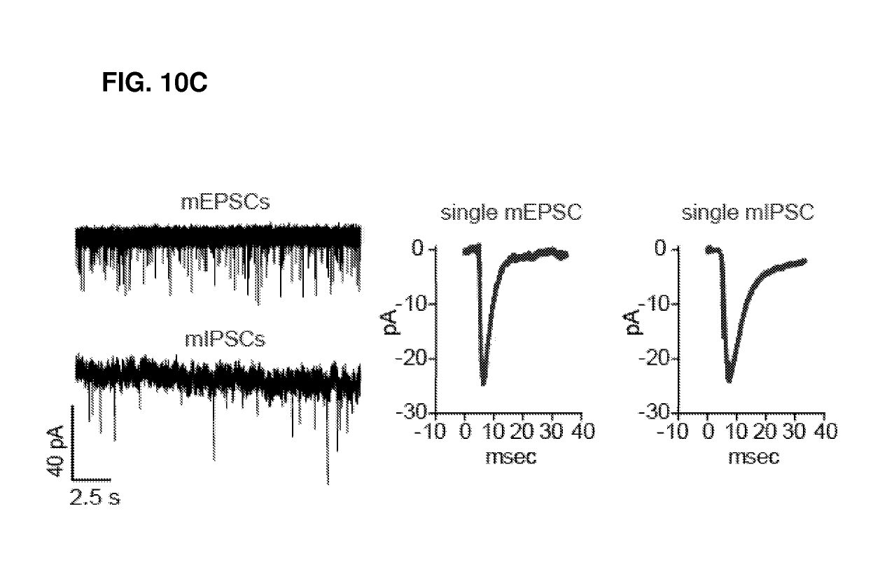

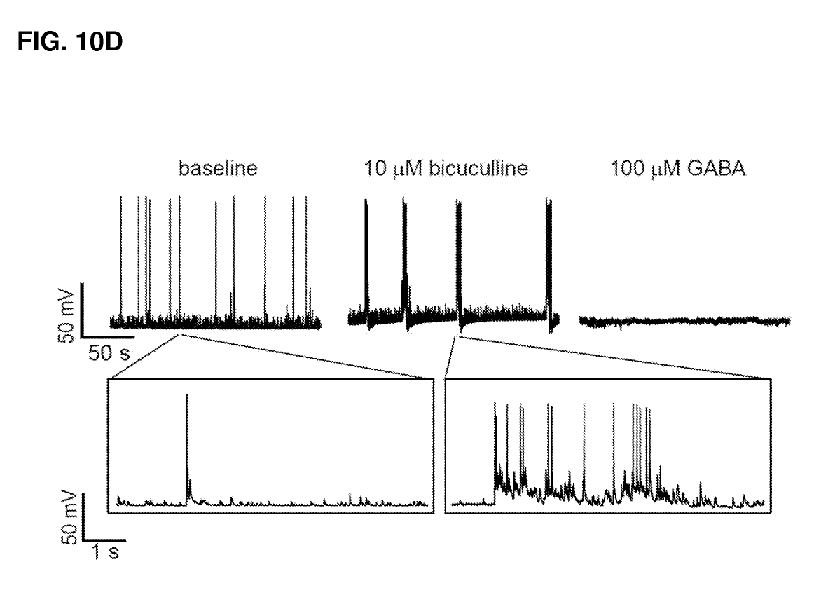

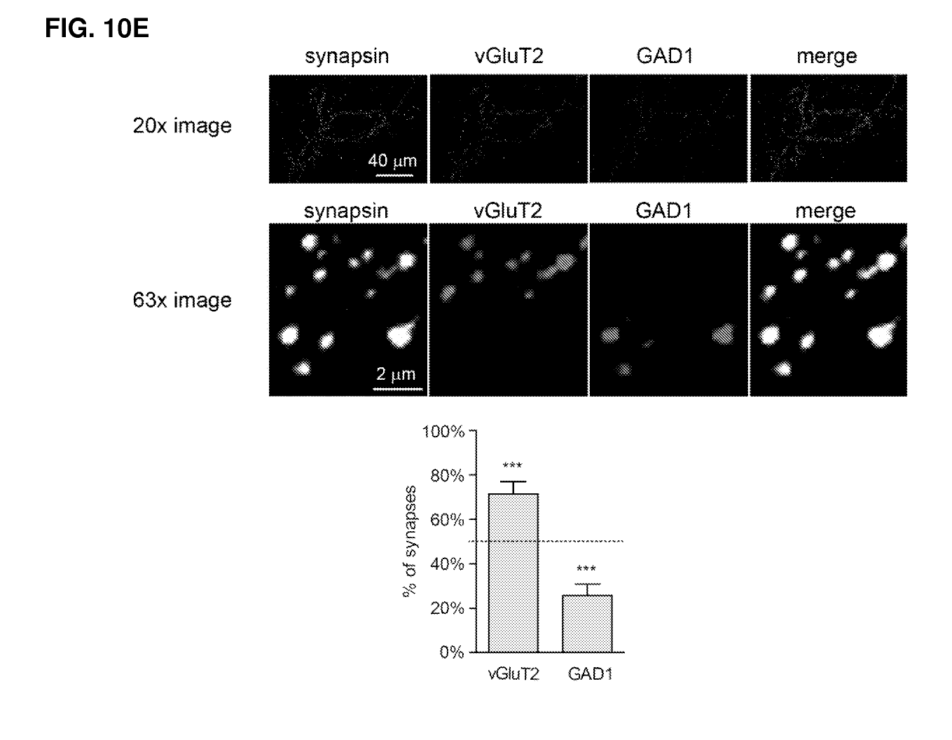

We have used a well-described differentiation model to produce mouse embryonic stem cell-derived neurons (ESNs) that express markers of glutamatergic and GABAergic identity, without evidence of other neurotransmitter systems. To evaluate the physiological relevance of ESNs for BoNT neurotoxicity studies, we first confirmed that ESNs developed morphological evidence of synapse formation (FIG. 10A). Neurotransmitter receptor-mediated currents were then measured at DIV 24+ using whole-cell patch clamp recordings in the presence of pharmacological agonists and antagonists of ionotropic glutamate receptors (iGluRs) and GABA receptors (GABARs). Perfusion with the excitatory neurotransmitter glutamate (FIG. 10B) produced a multiphasic inward current characteristic of a fast, desensitizing α-Amino-3-hydroxy-5-methyl-4-isoxazolepropionic acid (AMPA) receptor current and a non-desensitizing N-Methyl-D-aspartate (NMDA) receptor current, which terminated upon glutamate wash-out. Perfusion with the GABAR subtype A (GABARA) agonist muscimol resulted in a large desensitizing Cl-current, while the GABAR subtype B (GABARB)-specific agonist baclofen produced a sustained hyperpolarizing current (FIG. 10B). In the presence of tetrodotoxin (TTX), spontaneous excitatory and inhibitory monosynaptic currents with distinct decay kinetics were readily detected, confirming the presence of excitatory and inhibitory synapses (FIG. 10C). Addition of the GABARA antagonist bicuculline rapidly converted spontaneous network activity to synchronized epileptiform discharges with distinct bursting characteristics (FIG. 10D). Conversely, perfusion with the inhibitory neurotransmitter GABA silenced spontaneous network activity (FIG. 10D). Immunocytochemistry (ICC) co-localization studies indicated that 76.3±4.4% of synapses contained the presynaptic glutamatergic marker vGluT2. The remaining 23.7±4.5% of synapses contained the inhibitory presynaptic GABAergic marker GAD1/GAD67 (FIG. 10E). These data confirmed that DIV 24⁺ ESNs develop spontaneously active glutamatergic and GABAergic synapses and produce emergent network behaviors with characteristics of an excitatory/inhibitory (E/I) balance.

### Example 9

### BoNT/A Intoxication Silences Network Activity within 24 h

The effects of BoNT/A intoxication on synaptic activity were first evaluated by measuring the frequency of synaptically driven excitatory events in networked ESN cultures 24 h after addition of 20 pM BoNT/A (21.5 pM). This combination of time and dose was previously shown to cause nearly full cleavage of SNAP-25. Intoxication resulted in the significant reduction of excitatory events by 99.98±0.01% of controls (FIG. 11A). Intoxicated neurons were capable of producing repetitive action potentials (APs) in response to depolarizing current injection and resting membrane potentials were the same in vehicle controls and BoNT/A-intoxicated neurons (control=−87.2±3.5 mV; 24 h BoNT/A=−87.7±2.3 mV, n=24), indicating that loss of network activity was not attributable to impaired intrinsic electrical responses. This confirmed that BoNT/A intoxication of ESNs results in the same pathology that is responsible for clinical manifestations of botulism.

### Example 10

### BoNT/A Intoxication Causes Synaptic Inhibition

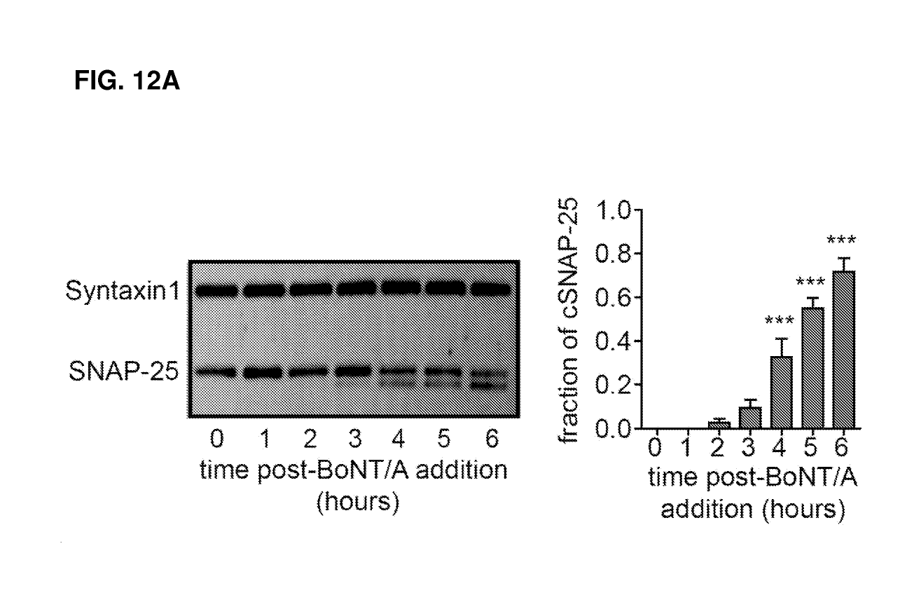

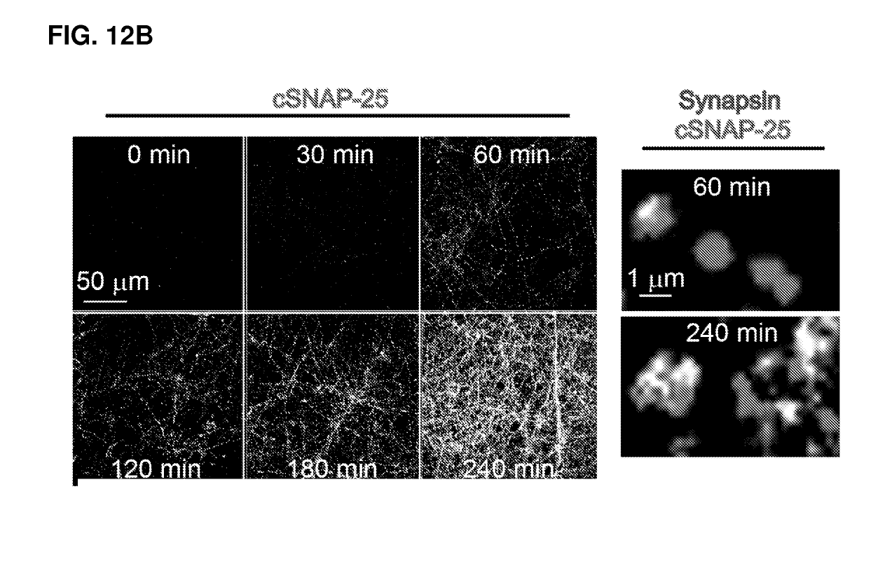

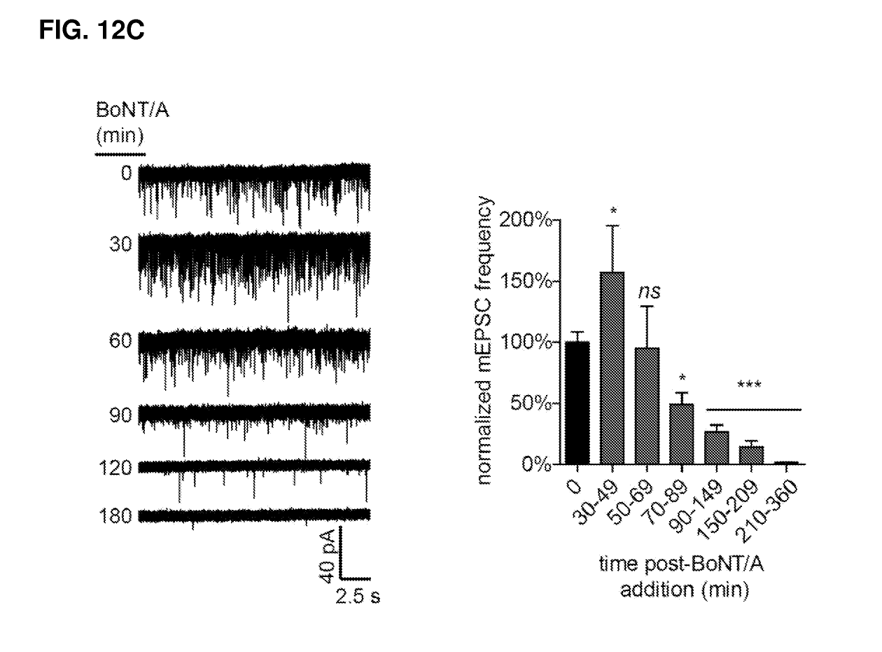

To explore the functional correlation between SNAP-25 cleavage and impairment of synaptic activity in ESNs, the spatiotemporal production of cleaved SNAP-25 (cSNAP-25) was examined by Western blots and immunocytochemistry. cSNAP-25 was first apparent in gel mobility shift assays at 2 hours, followed by a progressive increase through 6 hours (FIG. 12A). Co-localization studies using an antibody that was specific for the cleaved form of SNAP-25 demonstrated the accumulation of cSNAP-25 in presynaptic compartments from 60-240 min after exposure (FIG. 12B).

The association of cSNAP-25 with presynaptic terminals at early time points suggested that BoNT/A intoxication may rapidly alter synaptic activity. We developed an assay called the measured inhibition of synaptic activity (MISA) to quantify intoxication-induced changes in spontaneous synaptic activity. MISA is based on comparing the frequencies of spontaneous, miniature postsynaptic currents in intoxicated neurons to vehicle-treated controls, providing a direct measurement of the functional effect of intoxication on synaptic activity. Pharmacological antagonists can be used to gain additional resolution, allowing the effects of BoNT on glutamatergic versus GABAergic signaling to be readily distinguished.

MISA was used to quantify synaptic inhibition in DIV 24⁺ ESNs as a function of time following bath addition of BoNT/A (FIG. 12C). In this experiment, changes in miniature excitatory post-synaptic currents (mEPSCs) were quantified in a complex background containing both excitatory and inhibitory events, and therefore should be considered to be apparent mEPSC rates. BoNT/A addition caused a biphasic response in apparent mEPSCs, with an initial increase in frequency to 157.1±38.3% of controls at 30-49 min, followed by a decline in apparent mEPSCs until synaptic activity was essentially silenced between 210-360 min (FIG. 12B). Notably, the cleavage of 4.3% of total cellular SNAP-25 was sufficient reduce apparent mEPSC rates by 73.3±5.8%, indicating that cleavage of a subpopulation of SNAP-25 was sufficient to eliminate synaptic function (FIG. 12A, 12C).

### Example 11

### Inhibitory Synapses Undergo Accelerated Rates of Intoxication

The only known function of the light chain of BoNT/A (LC/A) is to target and cleave SNAP-25, preventing neurotransmitter release. Consequently, the finding that BoNT/A addition caused a transient increase in apparent mEPSCs was unexpected. One potential explanation is that apparent mEPSCs were measured in a complex background containing both excitatory and inhibitory postsynaptic signaling, such that concurrent inhibitory events would neutralize excitatory events. This led us to hypothesize that the initial increase in apparent mEPSCs was a consequence of the accelerated loss of inhibitory signaling rather than an increase in excitatory synaptic activity.

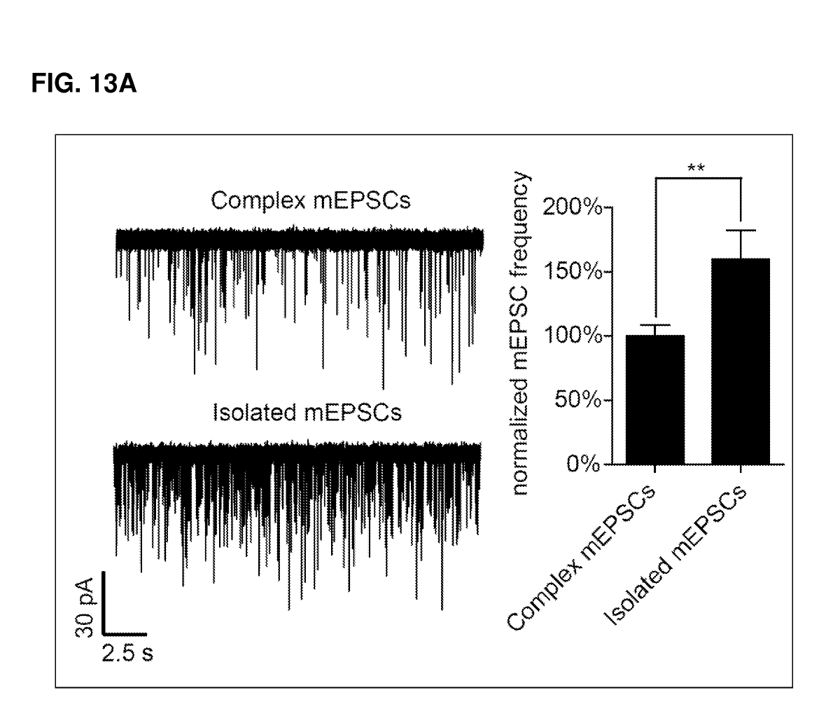

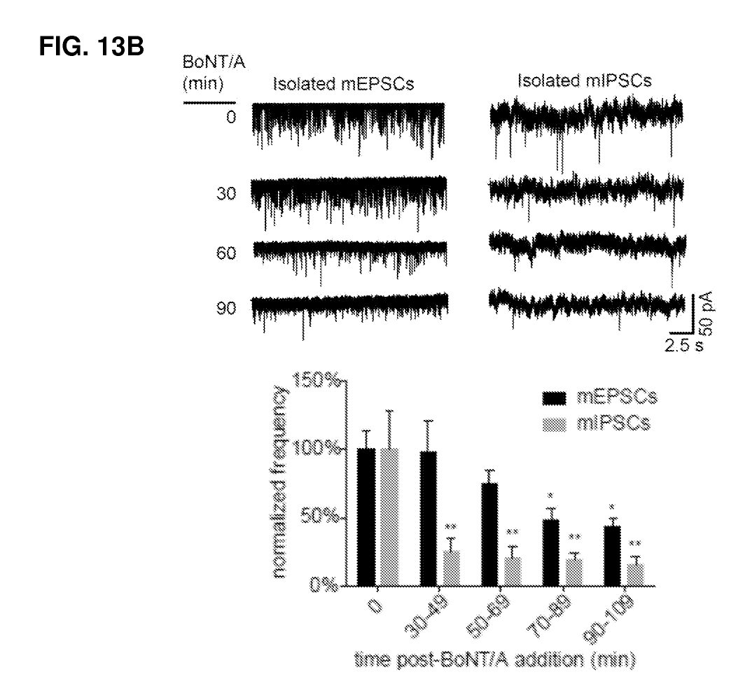

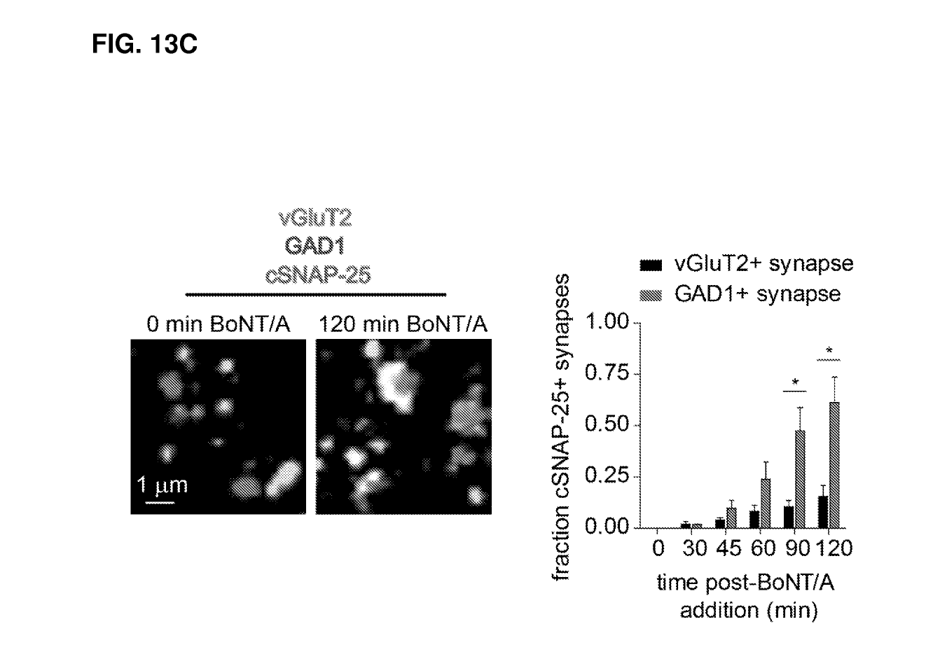

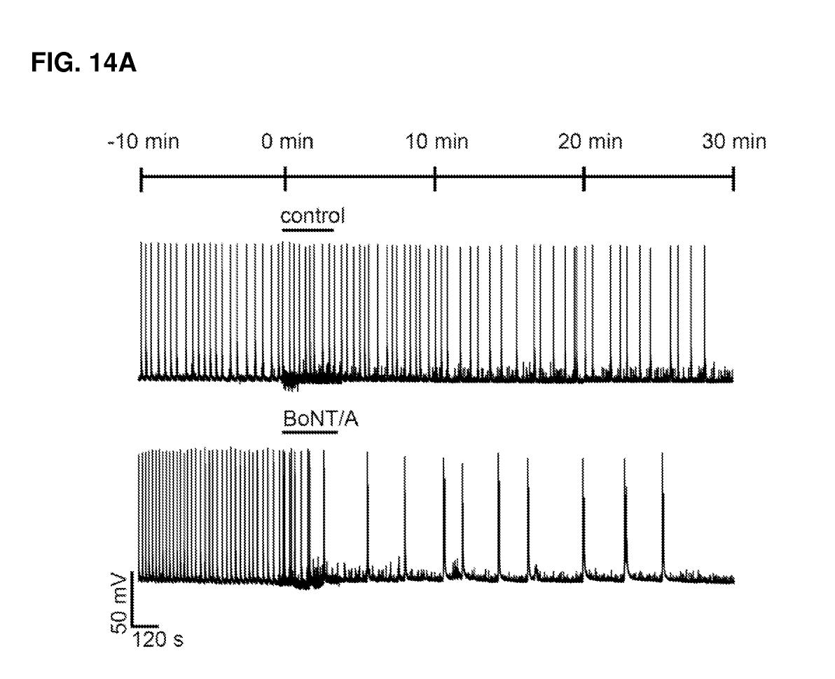

To determine whether inhibitory post-synaptic events could affect quantitation of mEPSCs, we compared the frequencies of apparent mESPCs to isolated mESPCs, measured in the presence of GABARA and GABARB antagonists. Isolated mEPSCs were ˜60% more frequent than apparent mEPSCs, (complex=6.41±0.5 Hz, isolated=9.78±1.4 Hz; FIG. 13A), suggesting that changes in mIPSC frequencies could alter measurements of apparent mEPSCs. We then quantified pharmacologically isolated mEPSCs or mIPSCs from 30-110 min after BoNT/A addition (FIG. 13B). Whereas isolated mIPSCs were significantly reduced to 25.3±10.1% of controls within 30 minutes, isolated mEPSCs did not undergo a significant change until 70 min, when they were reduced by 48.9±9.3%. Immunocytochemistry confirmed the preferential association of cSNAP-25 with GABAergic presynaptic markers at early time points (FIG. 13C). These data strongly support a model in which the transient increase in apparent mEPSCs is a consequence of the accelerated intoxication of inhibitory synapses.

### Example 12

### BoNT/A Addition Rapidly Leads to Network Disinhibition

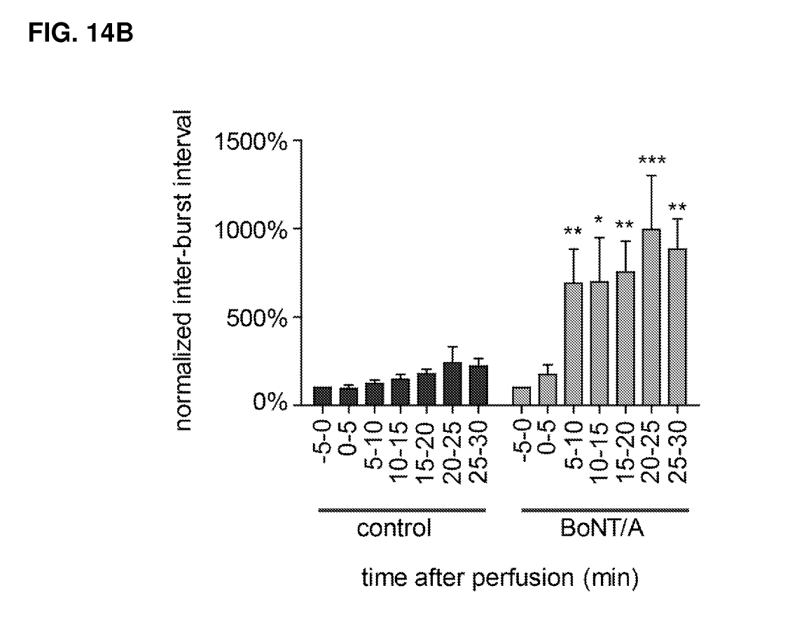

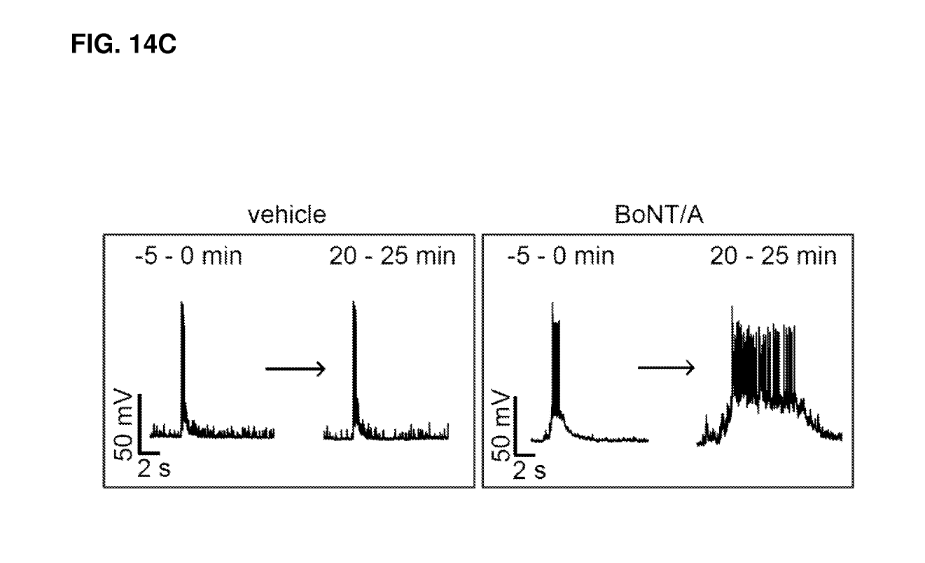

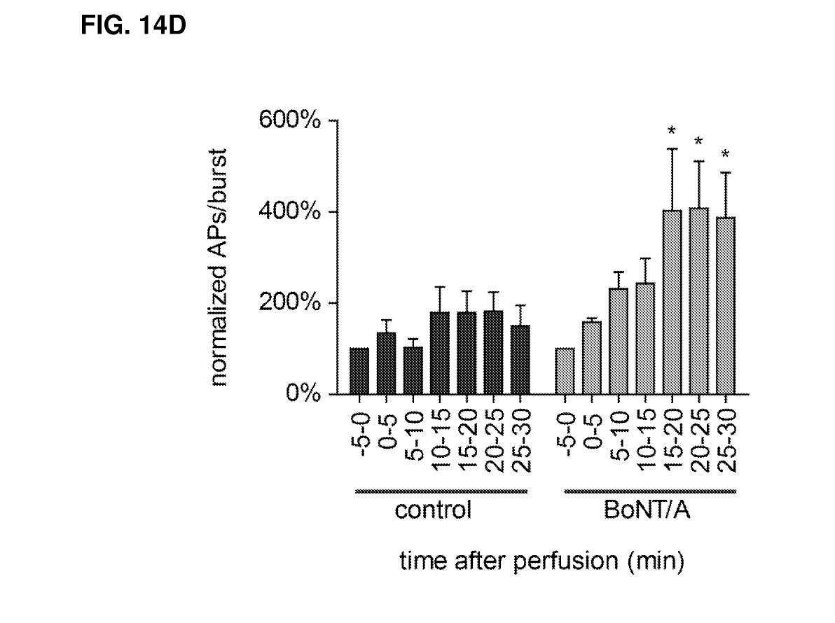

In networks containing excitatory and inhibitory inputs, even subtle changes in the E/I ratio can have large effects on network behavior. To determine the acute effects of intoxication on network-level activity, we characterized network behavior using long-term, whole-cell recordings prior to and following superfusion with BoNT/A. To increase the power and sensitivity of this approach, APs were first monitored by current clamp under basal conditions for at least 10 min prior to superfusion with either control solutions or BoNT/A. Spontaneous network activity was recorded for an additional 30 min and compared to the 5 min window immediately preceding superfusion. Neurons in dishes superfused with vehicle, 200 pM formalin-inactivated toxoid or 20 pM heat-inactivated BoNT/A displayed frequent, short bursts (FIG. 14A) that did not show significant changes in inter-burst interval (FIG. 14B) or bursting patterns (FIG. 14C-D) over the course of the experiment. In contrast, superfusion of BoNT/A to a final concentration of 20 pM caused a rapid transition to epileptiform bursting behavior within minutes, defined by large bursts with paroxysmal depolarizing shifts (FIG. 14A). Compared to time-match controls, BoNT/A-treated neurons exhibited significant increases in inter-burst intervals within 10 min (FIG. 14B) and APs per burst within 20 min (FIG. 14D).

The rate of apparent mEPSCs observed 30 min after BoNT/A intoxication (FIG. 12C) was nearly the same as the rate of isolated mEPSCs measured in control neurons (FIG. 13A).

The invention illustratively disclosed herein suitably may be practiced in the absence of any element, which is not specifically disclosed herein. It is apparent to those skilled in the art, however, that many changes, variations, modifications, other uses, and applications to the method are possible, and also changes, variations, modifications, other uses, and applications which do not depart from the spirit and scope of the invention are deemed to be covered by the invention, which is limited only by the claims which follow.

What is claimed is:

1. A method of detecting neurotoxin in a sample, the method comprising:
a. identifying a sample potentially exposed to neurotoxin;
b. providing an isolated neuron population, wherein the neuron population is capable of network synaptic activity;
c. measuring a first synaptic activity of the neuron population;
d. contacting the sample to the neuron population;
e. measuring a second synaptic activity of the neuron population;
f. comparing the measured synaptic activities; and,
g. correlating a change in synaptic activity with neurotoxin detection, wherein a decrease in synaptic activity is correlated with the presence of neurotoxin in the sample, wherein no change in synaptic activity is correlated with the absence of neurotoxin in the sample, and wherein an increase in synaptic activity is correlated with the absence of neurotoxin in the sample.

2. The method of claim 1 further comprising measuring a third synaptic activity at a time point that follows the measuring of the second synaptic activity.

3. The method of claim 2 further comprising measuring a fourth synaptic activity at a time point that follows the measuring of the third synaptic activity.

4. The method of claim 1, wherein the neuron population is selected from the group consisting of cortical neurons, hippocampal neurons, cerebellar neurons, basal ganglia neurons, spinal cord neurons and combinations thereof.

5. The method of claim 1, wherein the presence of botulinum neurotoxin is detected at a toxin level of less than 0.99 U/mL.

6. The method of claim 1, wherein the presence of botulinum neurotoxin is detected at a toxin level of at least 0.01 U/mL.

7. The method of claim 1, wherein the neurotoxin is selected from the group consisting of botulinum toxin, tetanus toxin, latrotoxin, shiga toxin, tetrodotoxin, conotoxin, and combinations thereof.

8. The method of claim 1, wherein the neurotoxin detected is selected from the group consisting of serotype /A, serotype /B, serotype /C, serotype /D, serotype /E, serotype /F, serotype /G, serotype /H, subtypes of serotype /A, subtypes of serotype /B, subtypes of serotype /C, subtypes of serotype /D, subtypes of serotype /E, subtypes of serotype /F, subtypes of serotype /G, subtypes of serotype /H, or combinations thereof.

9. The method of claim 1, wherein the sample is selected from the group consisting of a purified toxin, a partially purified toxin, or unpurified toxin.

10. The method of claim 1, wherein the sample is selected from the group consisting of a bulk toxin, a formulated toxin, a cosmetics toxin formulation, or a clinical toxin formulation.

11. The method of claim 1, wherein the sample is selected from the group consisting of a raw food, a cooked food, a partially cooked food, a processed food, or combinations thereof.

12. The method of claim 1, wherein the sample is taken from a subject.

13. The method of claim 12, wherein the sample is selected from the group consisting tissue, saliva, excretion, feces, blood, urine, or combinations thereof.

14. The method of claim 1 further comprising detecting the presence of neurotoxin in less than 3 hours after the contact of the sample with the neuron population.

15. The method of claim 1 further comprising detecting the presence of neurotoxin in less than 30 minutes after the contact of the sample with the neuron population.

16. A method of detecting synaptic preservation, the method comprising:
a. identifying a sample exposed to neurotoxin;
b. providing an isolated neuron population, wherein the neuron population is capable of network synaptic activity;
c. measuring a first synaptic activity of the neuron population;
d. contacting the sample to the neuron population;
e. measuring a second synaptic activity of the neuron population;
f. comparing the measured synaptic activities; and,
g. correlating a change in synaptic activity with synaptic preservation, wherein a decrease in synaptic activity is correlated with the presence of neurotoxin in the sample, wherein no change in synaptic activity is correlated with the presence of a neutralizing agent in the sample, and wherein an increase in synaptic activity is correlated with the presence of a neutralizing agent in the sample.

17. The method of claim 16 further comprising measuring subsequent synaptic activity at a time point that follows the measuring of the second synaptic activity.

18. A method of evaluating the efficacy of a neurotoxin neutralizing agent, the method comprising:
a. providing an isolated neuron population, wherein the neuron population is capable of network synaptic activity;
b. measuring a first synaptic activity of the neuron population;
c. contacting the neuron population with a neurotoxin;
d. measuring a second synaptic activity of the neuron population, wherein the second synaptic activity is measured after the neuron population is contacted with a neurotoxin;
e. contacting the neuron population with a neurotoxin neutralizing agent;
f. measuring a third synaptic activity of the neuron population, wherein the third synaptic activity is measured after the neuron population is contacted with a neurotoxin neutralizing agent;
g. comparing the measured synaptic activities; and,
h. correlating a change in synaptic activity with neurotoxin neutralization, wherein a decrease from the second synaptic activity measurement to subsequent synaptic activity measurement is correlated with the absence of neurotoxin neutralization, wherein no change in synaptic activity from the second synaptic activity measurement to subsequent synaptic activity measurement is correlated with neurotoxin neutralization, and wherein an increase from the second synaptic activity measurement to subsequent synaptic activity measurement is correlated with neurotoxin neutralization.

19. The method of claim 18 further comprising measuring subsequent synaptic activity at a time point that follows the measuring of the second synaptic activity.

20. The method of claim 18, wherein the neuron population is selected from the group consisting of cortical neurons, hippocampal neurons, cerebellar neurons, basal ganglia neurons, spinal cord neurons and combinations thereof.

21. The method of claim 18, wherein the neurotoxin neutralization agent is selected from the group consisting of antibody, small molecule, molecule, compound, and combinations thereof.

22. A method of identifying neurotoxins in a sample, the method comprising:
a. providing a sample exposed to neurotoxin;
b. providing an isolated neuron population, wherein the neuron population is capable of network synaptic activity;
c. contacting the neuron population with the sample;
d. measuring a first synaptic activity of the neuron population;
e. contacting the neuron population with a neurotoxin neutralizing agent;
f. measuring a second synaptic activity of the neuron population, wherein the second synaptic activity is measured after the neuron population is contacted with a neurotoxin;
g. comparing the measured synaptic activities; and,
h. correlating a change in synaptic activity with neurotoxin identity, wherein an increase in synaptic activity is correlated with the neurotoxin identity of the neutralizing agent specificity, wherein no change in synaptic activity is correlated with the neurotoxin identity of the neutralizing agent specificity, and wherein a decrease in synaptic activity is not correlated with the neurotoxin identity of the neutralizing agent specificity.

23. The method of claim 22, wherein the neutralizing agent specificity is selected from the group consisting of botulinum toxin, tetanus toxin, latrotoxin, shiga toxin, tetrodotoxin, conotoxin, and combinations thereof.

24. The method of claim 23, wherein the neutralizing agent specificity is selected from the group consisting of serotype /A, serotype /B, serotype /C, serotype /D, serotype /E, serotype /F, serotype /G, serotype /H, subtypes of serotype /A, subtypes of serotype /B, subtypes of serotype /C, subtypes of serotype /D, subtypes of serotype /E, subtypes of serotype /F, subtypes of serotype /G, subtypes of serotype /H, or combinations thereof.

25. The method of claim 22 further comprising measuring subsequent synaptic activity at a time point that follows the measuring of the second synaptic activity.
